(19) United States
(12) Patent Application Publication
(10) Pub. No.: US 2019/0380992 A1
(43) Pub. Date: Dec. 19, 2019

(54) Multiple Sclerosis Treatment Regimen Using Dimethyl Fumarate

(71) Applicant: Biogen MA Inc., Cambridge, MA (US)

(72) Inventor: Jitesh Rana, Brookline, MA (US)

(21) Appl. No.: 15/532,892

(22) PCT Filed: Dec. 3, 2015
(86) PCT No.: PCT/US2015/063765

§ 371 Date: Jun. 2, 2017

Related U.S. Application Data
(60) Provisional application No. 62/087,675, filed on Dec. 4, 2014.

(51) Int. Cl. A61K 31/225; A61K 9/20; A61K 9/28; A61K 9/48

(52) U.S. Cl. CPC A61K 31/225; A61K 9/4808; A61K 9/2846; A61K 9/2077

(57) ABSTRACT

Provided herein are methods of treating multiple sclerosis with dimethyl fumarate using an up-titration regimen. The methods provided herein are to reduce dimethyl fumarate-related gastro-intestinal adverse events in multiple sclerosis patients.

### CROSS-REFERENCES TO RELATED APPLICATIONS

This application claims the benefit of U.S. Provisional Patent Application No. 62/087,675, filed Dec. 4, 2014, which is incorporated herein by reference in its entirety.

### 1. FIELD

Provided herein are methods of treating multiple sclerosis with dimethyl fumarate using an up-titration regimen. The methods provided herein aim to reduce the fumarate-related gastro-intestinal adverse events in multiple sclerosis patients.

### 2. BACKGROUND

Multiple sclerosis (MS) is an autoimmune disease with the autoimmune activity directed against central nervous system (CNS) antigens. The disease is characterized by inflammation in parts of the CNS, leading to the loss of the myelin sheathing around neuronal axons (demyelination), axonal loss, and the eventual death of neurons, oligodendrocytes and glial cells. For a comprehensive review of MS and current therapies, see, e.g., McAlpine's Multiple Sclerosis, by Alastair Compston et al., 4th edition, Churchill Livingstone Elsevier, 2006.

More than 2.1 million people in the world suffer from MS, with roughly 400,000 of those living in the United States (see, e.g., Hanson et al., Patient Prefer Adherence, 2014, 8:415-422). It is one of the most common diseases of the CNS in young adults. MS is a chronic, progressing, disabling disease, which generally strikes its victims some time after adolescence, with diagnosis generally made between 20 and 40 years of age, although onset may occur earlier. Women are more likely than men to have the disease and MS itself is highly variable with symptoms and severity ranging from patient to patient (see, e.g., Ruggieri et al., Ther. Clin. Risk Manag., 2014, 10:229-239). The disease is not directly hereditary, although genetic susceptibility plays a part in its development. MS is a complex disease with heterogeneous clinical, pathological and immunological phenotype.

There are four major clinical types of MS: 1) relapsing-remitting MS (RR-MS), characterized by clearly defined relapses with full recovery or with sequalae and residual deficit upon recovery; periods between disease relapses characterized by a lack of disease progression; 2) secondary progressive MS (SP-MS), characterized by initial relapsing remitting course followed by progression with or without occasional relapses, minor remissions, and plateaus; 3) primary progressive MS (PP-MS), characterized by disease progression from onset with occasional plateaus and temporary minor improvements allowed; and 4) progressive relapsing MS (PR-MS), characterized by progressive disease onset, with clear acute relapses, with or without full recovery; periods between relapses characterized by continuing progression.

Clinically, the illness most often presents as a relapsing-remitting disease and, to a lesser extent, as steady progression of neurological disability. Relapsing-remitting MS (RR-MS) presents in the form of recurrent attacks of focal or multifocal neurologic dysfunction. Attacks may occur, remit, and recur, seemingly randomly over many years. Remission is often incomplete and as one attack follows another, a stepwise downward progression ensues with increasing permanent neurological deficit. The usual course of RR-MS is characterized by repeated relapses associated, for the majority of patients, with the eventual onset of disease progression. The subsequent course of the disease is unpredictable, although most patients with a relapsing-remitting disease will eventually develop secondary progressive disease. In the relapsing-remitting phase, relapses alternate with periods of clinical inactivity and may or may not be marked by sequalae depending on the presence of neurological deficits between episodes. Periods between relapses during the relapsing-remitting phase are clinically stable. On the other hand, patients with progressive MS exhibit a steady increase in deficits, as defined above and either from onset or after a period of episodes, but this designation does not preclude the further occurrence of new relapses.

MS pathology is, in part, reflected by the formation of focal inflammatory demyelinating lesions in the white matter, which are the hallmarks in patients with acute and relapsing disease. In patients with progressive disease, the brain is affected in a more global sense, with diffuse but widespread (mainly axonal) damage in the normal appearing white matter and massive demyelination also in the grey matter, particularly, in the cortex.

Salts of fumaric acid esters, in combination with dimethyl fumarate (DMF), such as present in FUMADERM®, have been proposed for the treatment of MS (see, e.g., Schimrigk et al., Eur. J. Neurol., 2006, 13(6):604-610; Drugs R&D, 2005, 6(4):229-30; U.S. Pat. No. 6,436,992). FUMADERM® contains dimethyl fumarate, calcium salt of ethyl hydrogen fumarate, magnesium salt of ethyl hydrogen fumarate, and zinc salt of ethyl hydrogen fumarate (see, e.g., Schimrigk et al., Eur. J. Neurol., 2006, 13(6):604-610).

Although currently there is no cure for MS, treatment options are available for patients with the disease. Currently available treatments typically focus on slowing the progression of the disease over time, improving quality of life, and reducing the number and severity of the symptoms of MS. For those patients with relapsing MS, common initial treatments have included interferon-beta (IFN-β) and glatiramer acetate (see, e.g., Fox et al., N. Engl. J. Med., 2012, 367(12):1087-1097; Erratum in: N. Engl. J. Med., 2012, 367(17):1673). Additional treatments have included natalizumab. In the past few years, fingolimod, teriflunomide, and delayed-release DMF were developed as oral treatments, which are expected to improve adherence to treatment (see, e.g., Cree B. A., Neurohospitalist, 2014, 4(2):63-65).

TECFIDERA®, dimethyl fumarate delayed-release capsules for oral use, was approved in 2013 by the U.S. Food and Drug Administration for the treatment of subjects with relapsing forms of multiple sclerosis. TECFIDERA® contains dimethyl fumarate (DMF), which has the following structure:

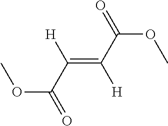

The first Phase 3 study, DEFINE (ClinicalTrials.gov identifier NCT00420212), demonstrated that DMF significantly reduced clinical relapses, accumulation of disability progression, and lesion number and volume compared with placebo after two years of treatment. See, e.g., Gold et al., N. Engl. J. Med. 2012(367):1098-1107. These findings were supported by the results of the second phase 3 study, CONFIRM (ClinicalTrials.gov identifier NCT00451451), which additionally evaluated subcutaneous glatiramer acetate as an active reference treatment (rater-blind). See, e.g., Fox et al., N. Engl. J. Med. 2012(367): 1087-1097.

DMF has demonstrated an acceptable safety profile in the DEFINE and CONFIRM studies. Gastrointestinal (GI) adverse events (AEs), for example nausea, vomiting, abdominal pain, and diarrhea, are the most common cause of AEs leading to discontinuation in patients initiating DMF.

Study 109HV321 (ClinicalTrials.gov identifier NCT01568112), to evaluate whether premedication with 325-mg micro-coated aspirin or a slow-titration dosing regimen reduces the incidence and/or severity of flushing and GI events following oral administration of DMF dosed at 240 mg BID in healthy volunteers, evaluated the effect of a 3-week titration. One arm of the study involved a 1-week fast-titration dosing schedule of a total daily dose of 240 mg DMF (120 mg BID) followed by a total daily dose of 480 mg DMF (240 mg BID) for 7 weeks. The second arm of the study involved a DMF slow-titration dosing schedule (4 increasing DMF doses over 4 weeks and maintaining the Week 4 dose (480 mg daily) until completion of the study (4 additional weeks). This study did not show benefit of the 3-week titration when compared to the standard 1-week titration (see Russell et al., Poster No. DX52 presented at: 5^{th} Cooperative Meeting of the CMSC-ACTRIMS, 2013, May 29-June 1; Orlando, Fla.).

There is a need in the art for methods of treating patients with DMF that reduce the DMF-related gastro-intestinal adverse events in multiple sclerosis patients.

Citation or discussion of information hereinabove shall not be deemed an admission that such is prior art to the present invention.

### 3. SUMMARY

The invention provides methods of treating a patient with multiple sclerosis (MS) including an up-titration protocol, wherein a starting dose of dimethyl fumarate (DMF) is lower than the maintenance dose and the daily dose of DMF is increased by 120 mg every 2 weeks.

In one embodiment, the invention provides a method of treating a human patient with MS comprising orally administering to the patient a pharmaceutical composition comprising DMF; wherein the administering step comprises administering a starting dose of 120 mg DMF daily for 2 weeks, followed by 240 mg DMF daily for 2 weeks, followed by 360 mg DMF daily for 2 weeks, followed by 480 mg DMF daily as a maintenance dose. In a specific aspect of such embodiment, the 240 mg DMF daily is 120 mg DMF BID. In a specific aspect of such embodiment, the 360 mg DMF daily is 240 mg DMF in the morning and 120 mg DMF in the evening. In a specific aspect of such embodiment, the 480 mg DMF daily is 240 mg DMF BID.

In a specific embodiment, the invention provides a method of treating a human patient with MS comprising orally administering to the patient a pharmaceutical composition comprising DMF; wherein the administering step comprises administering a starting dose of 120 mg DMF daily for 2 weeks, followed by 120 mg DMF BID for 2 weeks, followed by 360 mg DMF daily for 2 weeks, followed by 240 mg DMF BID as a maintenance dose; wherein the 360 mg DMF daily is 240 mg DMF in the morning and 120 mg DMF in the evening.

In a specific embodiment, administering of DMF as per a dosing regimen above results in a reduced incidence of gastrointestinal adverse events compared to a dosing regimen in which the dose is increased to 240 mg DMF BID immediately following a starting dose of 120 mg DMF BID for one week.

In a specific embodiment, the pharmaceutical composition comprises DMF and a pharmaceutically acceptable carrier.

In a specific embodiment, the pharmaceutical composition is in the form of a tablet or a capsule. In a specific embodiment, the pharmaceutical composition is in the form of an enterically coated tablet. In a specific embodiment, the pharmaceutical composition is in the form of a capsule containing enterically coated microtablets.

In a specific embodiment, the pharmaceutical composition consists essentially of DMF.

In a specific embodiment, the pharmaceutical composition does not contain a fumarate salt. In a specific embodiment, the pharmaceutical composition does not contain ethyl hydrogen fumarate salt. In a specific embodiment, the pharmaceutical composition does not contain ethyl hydrogen fumarate calcium salt, ethyl hydrogen fumarate magnesium salt, ethyl hydrogen fumarate zinc salt, and ethyl hydrogen fumarate copper salt.

In a specific embodiment, the multiple sclerosis is a relapsing form of multiple sclerosis.

### 3.1 Terminology

In order to provide a clear and consistent understanding of the specification and claims, the following definitions are provided:

As used herein, “a” or “an” means one or more unless otherwise specified or context clearly indicates otherwise.

Unless specifically stated or obvious from context, as used herein, the term “about” is understood as within a range of normal tolerance in the art, for example, within 20% of the stated value. As used herein, “about” a specific value also includes the specific value; for example, about 10%.

Other terms and/or abbreviations are provided below:

| Abbreviation or | |
|---|---|
| Specialist Term | Explanation |
| AE | Adverse event |
| AUC | Area under the curve |
| BID | Twice daily |
| Cmax | Maximum measured plasma concentration |
| DMF | Dimethyl fumarate |
| GI | Gastrointestinal |
| h, hr | Hour |
| MMF | Monomethyl fumarate |
| MS | Multiple Sclerosis |

### 4. BRIEF DESCRIPTION OF DRAWINGS

FIG. 1 depicts a schematic of the study design of Example 6.

### 5. DETAILED DESCRIPTION

The invention provides methods of treating a patient with multiple sclerosis (MS) including an up-titration protocol, wherein a starting dose of dimethyl fumarate (DMF) is lower than the maintenance dose and the daily dose of DMF is increased by 120 mg every 2 weeks.

In one embodiment, the invention provides a method of treating a human patient with MS comprising orally administering to the patient a pharmaceutical composition comprising DMF; wherein the administering step comprises administering a starting dose of 120 mg DMF daily for 2 weeks, followed by 240 mg DMF daily for 2 weeks, followed by 360 mg DMF daily for 2 weeks, followed by 480 mg DMF daily as a maintenance dose.

In a specific embodiment, the invention provides a method of treating a human patient with MS comprising orally administering to the patient a pharmaceutical composition comprising DMF; wherein the administering step comprises administering a starting dose of 120 mg DMF daily for 2 weeks, followed by 120 mg DMF BID for 2 weeks, followed by 360 mg DMF daily for 2 weeks, followed by 240 mg DMF BID as a maintenance dose; wherein the 360 mg DMF daily is 240 mg DMF in the morning and 120 mg DMF in the evening (or about 12 h apart).

In a specific embodiment, the methods of treating a patient with MS provided herein are to provide a reduction in the fumarate-related gastrointestinal (GI) adverse events, such as but not limited to nausea, vomiting, abdominal pain, and/or diarrhea, observed in some MS patients.

Accordingly, the invention provides methods of reducing fumarate-related GI adverse events in an MS patient, comprising administering DMF to the patient according to the treatment methods provided herein.

The methods of treatment provided herein can be to improve or reduce progression of a condition, symptom, disability, or parameter associated with MS

All of the various aspects, embodiments, and options disclosed herein can be combined in any and all variations. The compositions and methods provided are exemplary and are not intended to limit the scope of the claimed embodiments.

### 5.1 Active Agent for Use in the Methods Provided Herein

The active agent (i.e., drug) for use in the methods of treating MS and compositions of the invention is dimethyl fumarate (DMF).

In a specific embodiment, the DMF is administered in the form of a pharmaceutical composition.

### 5.2 Pharmaceutical Compositions

In one embodiment, DMF for use in the methods of the invention is contained in a pharmaceutical composition comprising DMF and a pharmaceutically acceptable carrier, i.e., a pharmaceutically acceptable excipient.

In a specific embodiment, DMF is administered as a pharmaceutical composition, wherein the pharmaceutical composition is TECFIDERA® DMF delayed release capsules.

In a specific embodiment, the pharmaceutical composition comprises DMF.

In a specific embodiment, the pharmaceutical composition comprises DMF as the only active agent except for breakdown products of DMF that appear under routine DMF handling or storage conditions.

In a specific embodiment, the pharmaceutical composition consists essentially of DMF.

In a specific embodiment, the pharmaceutical composition comprises DMF, with the proviso that a fumarate salt is not present in the pharmaceutical composition.

In a specific embodiment, the pharmaceutical composition comprises DMF, with the proviso that ethyl hydrogen fumarate salt is not present in the pharmaceutical composition.

In a specific embodiment, the pharmaceutical composition comprises DMF, with the proviso that ethyl hydrogen fumarate calcium salt, ethyl hydrogen fumarate magnesium salt, ethyl hydrogen fumarate zinc salt, and ethyl hydrogen fumarate copper salt are not present in the pharmaceutical composition.

In a specific embodiment, the pharmaceutical composition comprises DMF, with the proviso that no additional fumarate other than DMF and/or monomethyl fumarate (MMF) is present in the pharmaceutical composition.

In a specific embodiment, the pharmaceutical composition comprises DMF, with the proviso that the pharmaceutical composition contains no fumarate other than DMF and MMF.

In a specific embodiment, the pharmaceutical composition is not FUMADERM®, or does not contain all the active ingredients in FUMADERM®. FUMADERM® comprises the following active ingredients: dimethyl fumarate, calcium salt of ethyl hydrogen fumarate, magnesium salt of ethyl hydrogen fumarate, and zinc salt of ethyl hydrogen fumarate.

In a specific embodiment, DMF is the only active ingredient in the pharmaceutical composition. In a specific embodiment, DMF and MMF are the only active ingredients in the pharmaceutical composition.

In a specific embodiment, the pharmaceutical composition is an oral dosage form, e.g., a solid oral dosage form. In a specific embodiment, the pharmaceutical composition is a tablet, capsule, or capsule containing microtablets. Optionally, the tablet or microtablets are enterically coated. In a specific embodiment, the pharmaceutical composition is in the form of enterically coated tablets or microtablets (optionally contained in a capsule), which, once the enteric coating is dissolved in the gastro-intestinal tract, act as immediate release dosage forms.

In addition to administering the agent as a raw chemical, DMF may be administered as part of a pharmaceutical preparation containing suitable pharmaceutically acceptable carriers comprising excipients and auxiliaries which facilitate processing of the DMF into preparations which may be used pharmaceutically. For example, the preparations such as tablets, dragees, and capsules, as well as suitable solutions for administration orally, can contain from about 0.01 to 99 percent, preferably from about 0.25 to 75 percent of DMF together with the excipient.

In one embodiment, the composition is in the form of a dosage form, such that one composition provides the total DMF dose. In other embodiments, the dosage form contains multiple compositions to provide the total DMF dose. For example, a dosage form may contain multiple compacts, such as microtablets, to provide the desired total DMF dose.

If the dosage form contains multiple compacts, such as multiple microtablets, to provide the required total DMF dose, the compacts in the dosage form can be the same or can differ from one another. For example, the dosage form can contain two or more different microtablet types (e.g., the capsule can contain one group of microtablets coated with only an enteric coating and a second group of microtablets coated with only a seal coating, or one group coated with an enteric coating with a lower pH release and the other coated with an enteric coating with a higher pH release).

In some embodiments, the composition is placed in a capsule. In other embodiments, the composition, in the form of microtablets, is placed in a capsule. The microtablets can be enterically coated. The capsule can contain, for example, from about 30 microtablets to about 60 microtablets, from about 35 microtablets to about 55 microtablets, from about 30 to about 50 microtablets or from about 40 microtablets to about 50 microtablets (e.g., about 44, about 45, about 46, about 47, or about 48 microtablets).

In another specific embodiment, the pharmaceutical composition is a controlled, or sustained, release composition, optionally enterically coated.

The pharmaceutical preparations described herein are manufactured in a manner which is itself known, for example, by means of conventional mixing, granulating, dragee-making, dissolving, or lyophilizing processes. Thus, pharmaceutical preparations for oral use may be obtained by combining the fumarates with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

In one embodiment, the pharmaceutical preparations described herein comprise a capsule containing the active agent or pharmaceutical composition described herein in the form of an enteric-coated microtablet. The coating of the microtablet may be composed of different layers. The first layer may be a methylacrylic acid-methyl methacrylate copolymer/isopropyl solution which isolates the tablet cores from potential hydrolysis from the next applied water suspensions. The enteric coating of the tablet may then be conferred by an aqueous methacrylic acid-ethyl acrylate copolymer suspension.

In another embodiment, is provided a composition comprising DMF and one or more excipients, wherein a total amount of DMF in the composition ranges, for example, from about 43% w/w to about 95% w/w, based on the total weight of the composition, excluding the weight of any coating.

The total amount of DMF in the composition described herein can range, for example, from about 43% w/w to about 95% w/w, from about 50% w/w to about 95% w/w, from about 50% w/w to about 85% w/w, from about 55% w/w to about 80% w/w, from about 60% w/w to about 75% w/w, from about 60% w/w to about 70% w/w, or from about 65% w/w to about 70% w/w, based on the total weight of the composition, excluding the weight of any coating.

The composition described herein can comprise DMF, for example, in about 43% w/w, about 45% w/w, about 50% w/w, about 55% w/w, about 60% w/w, about 65% w/w, about 70% w/w, about 75% w/w, about 80% w/w, about 90% w/w, or about 95% w/w, based on the weight of the composition, excluding the weight of any coating. For example, the composition can contain about 65% to about 95% w/w (e.g., 65% w/w) of DMF.

Some or all of the DMF in the composition can have a particle size of 250 microns or less. For example, and without being limiting, at least 80%, at least 90%, at least 95%, at least 97%, or at least 99% of the DMF in the composition can have a particle size of 250 microns or less. Particle size can be measured, for example, by sieve analysis, air elutriation analysis, photoanalysis, electrical counting methods, electroresistance counting methods, sedimentation techniques, laser diffraction methods, acoustic spectroscopy, or ultrasound attenuation spectroscopy. In one embodiment, the particle size is measured using laser diffraction methods.

The composition described herein can comprise a total amount of excipient(s), for example, in an amount of about 5.0% w/w to about 57% w/w, based on the total weight of the composition, excluding the weight of any coating.

The composition described herein can comprise a total amount of excipient(s) in an amount ranging, for example, from about 5% w/w to about 57% w/w, from about 15% w/w to about 57% w/w, from about 20% w/w to about 57% w/w, from about 25% w/w to about 57% w/w, from about 30% w/w to about 57% w/w, from about 35% w/w to about 57% w/w, from about 40% to about 57% w/w, from about 45% w/w to about 57% w/w, from about 50% w/w to about 57% w/w, from about 55% w/w to about 57% w/w, from about 5% w/w to about 55% w/w, from about 5% w/w to about 50% w/w, from about 5% w/w to about 45% w/w, from about 5% w/w to about 40% w/w, from about 5% w/w to about 35% w/w, from about 5% w/w to about 30% w/w, from about 5% w/w to about 25% w/w, from about 5% w/w to about 20% w/w, from about 5% w/w to about 15% w/w, from about 15% w/w to about 55% w/w, from about 20% w/w to about 50% w/w, from about 25% w/w to about 45% w/w, from about 30% w/w to about 40% w/w, from about 35% to about 40% w/w, based on the total weight of the composition, excluding the weight of any coating.

The excipient(s) can be, for example, one or more selected from the group consisting of a filler (or a binder), a glidant, a disintegrant, a lubricant, or any combination thereof.

The number of excipients that can be included in a composition is not limited.

Examples of fillers or binders include, but are not limited to, ammonium alginate, calcium carbonate, calcium phosphate, calcium sulfate, cellulose, cellulose acetate, compressible sugar, confectioner's sugar, dextrates, dextrin, dextrose, erythritol, ethylcellulose, fructose, glyceryl palmitostearate, hydrogenated vegetable oil type I, isomalt, kaolin, lactitol, lactose, mannitol, magnesium carbonate, magnesium oxide, maltodextrin, maltose, mannitol, medium chain triglycerides, microcrystalline cellulose, polydextrose, polymethacrylates, simethicone, sodium alginate, sodium chloride, sorbitol, starch, sucrose, sugar spheres, sulfobutylether beta-cyclodextrin, talc, tragacanth, trehalsoe, polysorbate 80, and xylitol. In one embodiment, the filler is microcrystalline cellulose. The microcrystalline cellulose can be, for example, PROSOLV SMCC® 50, PROSOLV SMCC® 90, PROSOLV SMCC® HD90, PROSOLV SMCC® 90 LM, and any combination thereof.

Examples of disintegrants include, but are not limited to, hydroxypropyl starch, alginic acid, calcium alginate, carboxymethylcellulose calcium, carboxymethylcellulose sodium, powdered cellulose, chitosan, colloidal silicon dioxide, croscarmellose sodium, crospovidone, docusate sodium, guar gum, hydroxypropyl cellulose, low substituted hydroxypropyl cellulose, magnesium aluminum silicate, methylcellulose, microcrystalline cellulose, polacrilin potassium, povidone, sodium alginate, sodium starch glycolate, starch, and pregelatinized starch. In one embodiment, the disintegrant is croscarmellose sodium.

Examples of glidants include, but are not limited to, calcium phosphate, calcium silicate, powdered cellulose, magnesium silicate, magnesium trisilicate, silicon dioxide, talcum and colloidal silica, and colloidal silica anhydrous. In one embodiment, the glidant is colloidal silica anhydrous, talc, or a combination thereof.

Examples of lubricants include, but are not limited to, canola oil, hydroxyethyl cellulose, lauric acid, leucine, mineral oil, poloxamers, polyvinyl alcohol, talc, oxtyldodecanol, sodium hyaluronate, sterilizable maize starch, triethanolamine, calcium stearate, magnesium stearate, glycerin monostearate, glyceryl behenate, glyceryl palmitostearate, hydrogenated castor oil, hydrogenated vegetable oil type I, light mineral oil, magnesium lauryl sulfate, medium-chain triglycerides, mineral oil, myristic acid, palmitic acid, poloxamer, polyethylene glycol, potassium benzoate, sodium benzoate, sodium chloride, sodium lauryl sulfate, stearic acid, talc, and zinc stearate. In one embodiment, the lubricant is magnesium stearate.

The composition described herein can comprise a total amount of filler(s) in an amount ranging from about 3.5% w/w to about 55% w/w of the composition, based on the total weight of the composition, excluding the weight of any coating.

The filler(s) can be comprised in the composition described herein, for example, in a total amount, for example, ranging from about 5% w/w to about 55% w/w, from about 10% w/w to about 55% w/w, from about 15% w/w to about 55% w/w, from about 20% w/w to about 55% w/w, from about 25% w/w to about 55% w/w, from about 30% w/w to about 55% w/w, from about 35% w/w to about 55% w/w, from about 40% w/w to about 55% w/w, from about 3.5% w/w to about 55% w/w, from about 3.5% to about 50%, from about 3.5% w/w to about 40% w/w, from about 3.5% w/w to about 30% w/w, from about 3.5% w/w to about 25% w/w, from about 3.5% w/w to about 20% w/w, from about 3.5% w/w to about 15% w/w, from about 15% w/w to about 40% w/w, from about 20% w/w to about 35% w/w, or from about 25% w/w to about 30% w/w, based on the total weight of the composition, excluding the weight of any coating.

The filler(s) can be comprised in the composition, for example, in a total amount of about 5% w/w, about 7% w/w, about 10% w/w, about 12% w/w, about 14% w/w, about 16% w/w, about 18% w/w, about 20% w/w, about 22% w/w, about 24% w/w, about 26% w/w, about 28% w/w, about 30% w/w, about 32% w/w, about 34% w/w, about 36% w/w, about 38% w/w, about 40% w/w, about 42% w/w, about 44% w/w, about 46% w/w, about 48% w/w, about 50% w/w, about 52% w/w, about 54% w/w, or about 55% w/w, based on the total weight of the composition, excluding the weight of any coating.

The composition described herein can comprise a total amount of disintegrant(s), for example, in an amount ranging from about 0.2% w/w to about 20% w/w, based on the total weight of the composition, excluding the weight of any coating.

The disintegrant(s) can be contained in the composition, for example, in a total amount ranging from about 0.2% w/w to about 19% w/w, about 0.2% w/w to about 15% w/w, about 0.2% w/w to about 12% w/w, about 0.2% w/w to about 6% w/w, about 0.2% w/w to about 5% w/w, about 0.2% w/w to about 4% w/w, about 0.2% w/w to about 3% w/w, about 0.2% w/w to about 2% w/w, about 0.2% w/w to about 20% w/w, about 3% w/w to about 20% w/w, about 4% w/w to about 20% w/w, about 5% w/w to about 20% w/w, about 6% w/w to about 20% w/w, about 7% w/w to about 20% w/w, about 8% w/w to about 20% w/w, about 9% w/w to about 20% w/w, about 2% w/w to about 20% w/w, or about 3% w/w to about 20% w/w, based on the weight of the composition, excluding the weight of any coating.

The disintegrant(s) can be contained in the composition, for example, in a total amount of about 1% w/w, about 2% w/w, about 3% w/w, about 4% w/w, about 5% w/w, about 6% w/w, about 7% w/w, about 8% w/w, about 9% w/w, about 10% w/w, about 12% w/w, about 14% w/w, about 16% w/w, about 18% w/w, or about 19% w/w, based on the total weight of the composition, excluding the weight of any coating.

The glidant(s) can be contained in the composition, for example, in a total amount ranging from about 0.1% w/w to about 9.0% w/w, based on the total weight of the composition, excluding the weight of any coating.

The glidant(s) can be contained in the composition, for example, in a total amount ranging from about 0.1% w/w to about 9.0% w/w, from about 0.1% w/w to about 8% w/w, from about 0.1% w/w to about 6% w/w, from about 0.1% w/w to about 4% w/w, from about 0.1% w/w to about 2.8% w/w, from about 0.1% w/w to about 2.6% w/w, from about 0.1% w/w to about 2.4% w/w, from about 0.1% w/w to about 2.2% w/w, from about 0.1% w/w to about 2.0% w/w, from about 0.1% w/w to about 1.8% w/w, from about 0.1% w/w to about 1.6% w/w, from about 0.1% to about 1.4% w/w, from about 0.1% w/w to about 1.2% w/w, from about 0.1% w/w to about 1.0% w/w, from about 0.1% w/w to about 0.8% w/w, from about 0.1% w/w to about 0.4% w/w, from about 0.2% w/w to about 3.0% w/w, from about 0.4% w/w to about 3.0% w/w, from about 0.6% w/w to about 3.0% w/w, from about 0.8% w/w to about 3.0% w/w, from about 1.0% w/w to about 3.0% w/w, from about 1.2% w/w to about 9.0% w/w, from about 1.4% w/w to about 9.0% w/w, from about 1.6% w/w to about 9.0%, from about 1.8% w/w to about 9.0% w/w, from about 2.0% w/w to about 9.0% w/w, from about 2.2% w/w to about 9.0% w/w, from about 2.4% w/w to about 9.0% w/w, from about 2.6% w/w to about 9.0% w/w, from about 2.8% w/w to about 9.0% w/w, from about 3.0% w/w to about 9.0% w/w, from about 4.0% w/w to about 9.0% w/w, from about 5.0% w/w to about 9.0% w/w, from about 6.0% w/w to about 9.0% w/w, from about 7.0% w/w to about 9.0% w/w, from about 8.0% w/w to about 9.0% w/w, from about 0.5% w/w to about 2.5% w/w, or from about 1.0% w/w to about 2.0% w/w, based on the total weight of the composition, excluding the weight of any coating.

The glidant(s) can be contained in the composition, for example, in a total amount of about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.2% w/w, about 1.4% w/w, about 1.6% w/w, about 1.8% w/w, about 2.0% w/w, about 2.2% w/w, about 2.4% w/w, about 2.6% w/w, about 2.8% w/w, about 3% w/w, about 4% w/w, about 5% w/w, about 6% w/w, about 7% w/w, about 8% w/w, or about 9% w/w, based on the total weight of the composition, excluding the weight of any coating.

The lubricant(s) can be contained in the composition, for example, in a total amount ranging from about 0.1% w/w to about 3.0% w/w, based on the total weight of the composition, excluding the weight of any coating.

The lubricant(s) can be contained in the composition, for example, in a total amount ranging from about 0.1% w/w to about 2% w/w, about 0.1% w/w to about 1% w/w, from about 0.1% w/w to about 0.7% w/w, from about 0.1% w/w to about 0.6% w/w, from about 0.1% w/w to about 0.5% w/w, from about 0.1% w/w to about 0.4% w/w, from about 0.1% w/w to about 0.3% w/w, from about 0.1% w/w to about 0.2% w/w, from about 0.2% w/w to about 3.0% w/w, from about 0.3% w/w to about 3.0% w/w, from about 0.4% w/w to about 3.0% w/w, from about 0.5% w/w to about 3.0% w/w, from about 0.6% w/w to about 3.0% w/w, from about 0.7% w/w to about 3.0% w/w, from about 0.8% w/w to about 3.0% w/w, from about 0.9% w/w to about 3.0% w/w, from about 1% w/w to about 3.0% w/w, from about 2% w/w to about 3% w/w, from about 0.2% w/w to about 0.7% w/w, from about 0.3% w/w to about 0.6% w/w, or from about 0.4% w/w to about 0.5% w/w, based on the total weight of the composition, excluding the weight of any coating.

The lubricant(s) can be contained in the composition, for example, in a total amount of about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 2.0% w/w, or about 3.0% w/w, based on the total weight of composition, excluding the weight of any coating.

In some embodiments, for example, the composition described herein comprises one or more fillers in a total amount ranging from about 3.5% w/w to about 55% w/w, one or more disintegrants in a total amount ranging from about 0.2% w/w to about 20% w/w, one or more glidants in a total amount ranging from about 0.1% w/w to about 9.0% w/w, and one or more lubricants in a total amount ranging from about 0.1% w/w to about 3.0% w/w.

In some embodiments, for example, the composition described herein comprises a filler, a disintegrant, a glidant, and a lubricant. In some embodiments, the filler is microcrystalline cellulose, the disintegrant is croscarmellose sodium, the glidant is colloidal silica anhydrous, and the lubricant is magnesium stearate. In other embodiments, the filler is microcrystalline cellulose, the disintegrant is croscarmellose sodium, the glidant is a combination of colloidal silica anhydrous and talc, and the lubricant is magnesium stearate.

The ingredients in the composition described herein can be, for example, homogeneous or heterogeneously mixed. The composition ingredients can be, for example, mixed by any known method including shaking, stirring, mixing with forced air, mixing in a spinning container, and the like. The composition ingredients can be, for example, mixed all at once, or with progressive addition of one or more ingredients. The composition ingredients can be mixed in any order, for example, individually, in groups, or as a blend of all of the ingredients. For example, the glidant(s) can be mixed with the DMF and/or disintegrant(s) prior to mixing with any or all of the filler(s) and/or lubricants. The blend can also be prepared by mixing DMF, disintegrant(s) (e.g., croscarmellose sodium) and a portion of binder (e.g., microcrystalline cellulose) before then passing through a screen or sieve. The remaining binder can be mixed with lubricant(s) (e.g., magnesium stearate) before passing through a screen or sieve. These two mixtures can then be combined and mixed before adding glidant(s) (e.g., silica colloidal anhydrous). The glidant(s) can also be added to one or both of the aforementioned mixtures before they are combined and mixed to produce the final blend.

The composition described herein can have a flowability index, for example, ranging from about 8 mm to about 24 mm. For example, the flowability index can range from about 12 mm to about 22 mm, from about 12 mm to about 20 mm, from about 12 mm to about 18 mm, from about 12 mm to about 16 mm, from about 12 mm to about 14 mm, from about 14 mm to about 24 mm, from about 16 mm to about 24 mm, from about 18 mm to about 24 mm, from about 20 mm to about 24 mm, from about 22 mm to about 24 mm, from about 14 mm to about 22 mm, or from about 16 mm to about 20 mm.

The flowability index can be, for example, less than 18 mm (e.g., about 8 mm, about 12 mm, about 14 mm, about 16 mm) with an amount of glidant(s) ranging from about 0.1% w/w to about 2.0% w/w (e.g., 1.0% w/w).

The flowability index can be measured, for example, on a FLODEX device (manufactured by Hanson Research). The following protocol, for example, can be employed: A powder sample (e.g., 50 g) is loaded into the cylinder on the FLODEX device such that the powder is within about 1 cm from the top of the cylinder. A minimum of 30 seconds is allowed to pass before testing commences. Starting with a 16 mm flow disk, the release lever is slowly turned until the closure drops open without vibration. The test is positive when the open hole at the bottom is visible when looking down from the top. If a positive result is obtained, the test is repeated with smaller and smaller disk holes until the test is negative. For negative results, the size of the flow disk hole is increased until the test is positive. The flowability index is the diameter of the smallest hole through which the sample will pass for three successive tests.

The composition can have, for example, a compressibility index ranging from about 15% to about 28%. The compressibility index can range, for example, from 17% to about 28%, from about 19% to about 28%, from about 21% to about 28%, from about 23% to about 28%, from about 25% to about 28%, from about 15% to about 26%, from about 15% to about 24%, from about 15% to about 22%, from about 15% to about 20%, from about 15% to about 18%, from about 17% to about 26%, from about 19% to about 24%, or from about 20% to about 22%.

The composition can have a compressibility index, for example, of about 16%, about 17%, about 18%, about 19%, about 20%, about 21%, about 22%, about 23%, about 24%, about 25%, about 26%, or about 27%.

The compressibility index can be defined, for example, by the formula: (((Vₒ−V_{f})/Vₒ)×100%) where Vₒ is unsettled apparent volume of the particle and V_{f} is the final tapped volume of the powder. The compressibility index can be determined, for example, as follows: powder is placed in a container and the powder's unsettled apparent volume (Vₒ) is noted. Next, the powder is tapped until no further volume changes occur. At this point, the final tapped volume of the powder is measured (V_{f}). The compressibility index is then calculated by using the formula above.

In some embodiments, the composition can be in the form of a powder (not compressed) or a compact (compressed). The shape of the compact is not limited and can be, for example, cubic, spherical, or cylindrical (e.g., disc-shaped).

The compact can be, for example, in the form of tablets, caplets, or microtablets. The compact can be prepared by any means known in the art. For example, if the compact is in the form of microtablets, the microtablets can be made by compressing the composition described above using any known method, such as using a rotary tablet press equipped with a multi-tip tooling and having concave tips.

Multi-tip tableting tools, for example, can be used. For example, a multi-tip tool having from about 16 tips to about 40 tips using, for example, about 2 mm diameter tips. In this situation, applied compressing force can be expressed as an average kN/tip. For example, an applied compressing force of 2 kN used with a 16 multi-tip tool yields an applied compressing force of about 0.125 kN/tip. Similarly, an applied compressing force of about 15 kN used with a 16 multi-tip tool yields an applied compressing force of about 0.94 kN per tip.

The microtablets can have a mean diameter (excluding any coatings), for example, ranging from about 1 mm to about 3 mm. For example, the microtablets can have a mean diameter ranging from about 1 mm to about 2.5 mm. The microtablets can have a mean diameter of about 1.0 mm, about 2.0 mm, or about 3.0 mm.

Compact tensile strength can be determined by any means known in the art. For example, the following protocol could be employed. First, compact(s) are compressed to about 360 mg weight using an instrumented rotary tablet press equipped to measure compression force with round flat tooling of approximately 10 mm diameter. Next, measure the diametrial crushing strengthusing a suitable tablet hardness tester and then calculate tensile strength by the procedure reported by Newton (Newton, J. M., Journal of Pharmacy and Pharmacology, 26: 215-216 (1974)). See also Pandeya and Puri, KONA Powder and Particle Journal, 30: 211-220 (2013), Jarosz and Parrott, J. Pharm. Sci. 72(5):530-535 (1983), and Podczeck, Intl. J. Pharm. 436:214-232 (2012).

The composition described herein, in the form of a compact, can have a tensile strength equal to or greater than 1.5 MPa at an applied or compaction pressure of about 100 MPa. For example, the tensile strength can range from about 2.0 to about 5.0 MPa (e.g., from about 2.5 to about 4.5 MPa, from about 3.0 to about 4.5 MPa or from about 3.5 to about 4.5 MPa) at an applied or compaction pressure of about 100 MPa. For example, the tensile strength can be about 4.0 MPa at an applied or compaction pressure of about 100 MPa.

The compact in the form of one or more microtablets produced using 16 multi-tip tooling can have a hardness or breaking strength or crushing strength ranging from about 8 N to about 35 N when the microtablet is formed by a compression force ranging from 2 kN to about 15 kN and the microtablet has a 2 mm diameter, a thickness of 2 mm, and a 1.8 mm radius of the convex surface. In one embodiment, microtablets each having a 2 mm diameter, a thickness of 2 mm, and a 1.8 mm radius of the convex surface have a hardness ranging from about 17 N to about 24 N for a compression force of about 4 kN to about 7 kN. The hardness can be, for example, of from about 23 N to about 27 N (e.g., about 24 N, about 25 N, or about 26 N) for a compression force of about 10 kN to about 15 kN. Hardness or breaking strength or crushing strength can be determined for example, using an Erweka tester or a Schleuniger tester as described in Lachman, L. et al., The Theory & Practice of Industrial Pharmacology (3rd ed. 1986), p. 298.

In some embodiments, the composition can be optionally coated or partially coated by one or more coatings. The coating(s) can be pH independent or pH dependent. The coating(s) can be, for example, enteric coatings, seal coatings, or combinations of enteric coatings and seal coatings.

The seal coating can contain, for example, one or more plasticizers, one or more copolymers, one or more polymers, or combinations thereof.

The plasticizer can be, for example, one or more of acetyltributyl citrate, acetyltriethyl citrate, benzyl benzoate, cellulose acetate phthalate, chlorbutanol, dextrin, dibutyl phthalate, dibutyl secacate, diethyl phthalate, dimethyl phthalate, glycerin, glycerin monostearate, hypromellose phthalate, mannitol, mineral oil an lanolin alcohols, palmitic acid, polyethylene glycol, polyvinyl acetate phthalate, propylene glycol, 2-pyrrolidone, sorbitol, stearic acid, triacetin, tributyl citrate, triethanolamine, and triethyl citrate.

The copolymer can be, for example, a methacrylic acid-methacrylate copolymer or a methacrylic acid-ethylacrylate copolymer.

Additionally, the seal coating can contain one or more polymers, for example, cellulose derivatives such as hydroxyethyl cellulose, hydroxypropyl cellulose, hydroxypropyl and methylcellulose, polyvinylpyrrolidone, a polyvinylpyrrolidone/vinyl acetate copolymer, ethyl cellulose, and ethyl cellulose aqueous dispersions (AQUACOAT®, SURELEASE®), EUDRAGIT® RL 30 D, OPADRY®, EUDRAGIT® S, EUDRAGIT® L, and the like.

If present in the seal coating, the total amount of one or more copolymer(s) and/or one or more polymer(s) in the seal coating can range, for example, from a positive amount greater than 0% w/w to about 100% w/w, based on the weight of the seal coating. The amount of one or more copolymer(s) and/or one or more polymer(s) in the seal coating can range, for example, from about 10% w/w to about 100% w/w, from about 20% w/w to about 100% w/w, from about 30% w/w to about 100% w/w, from about 40% w/w to about 100% w/w, from about 50% w/w to about 100% w/w, from about 60% w/w to about 100% w/w, from about 70% w/w to about 100% w/w, from about 80% w/w to about 100% w/w, or from about 90% w/w to about 100% w/w, based on the weight of the seal coating.

The amount of one or more copolymer(s) and/or one or more polymer(s) in the seal coating can be, for example, about 10% w/w, about 20% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w, about 90% w/w, or about 95% w/w, based on the weight of the seal coating.

If present in the seal coating, the mean amount of plasticizer in the seal coating can range, for example, from a positive amount greater than 0% w/w to about 70% w/w, based on the weight of the seal coating.

The enteric coating can contain, for example, one or more plasticizers, one or more fillers, one or more lubricants, one or more copolymers, one or more polymers, and any combinations thereof.

The plasticizer(s) in the enteric coat can be the same or different than any plasticizer(s) in a seal coat, if present, and can be one of more of the plasticizers listed above.

The filler(s) in the enteric coat can be the same or different than any filler(s) in the composition. Additionally, the filler(s) in the enteric coat can be the same or different than any filler(s) in a seal coat, if present, and can be one or more of the fillers listed above.

The lubricant(s) in the enteric coat can be the same or different than any lubricant(s) in the composition. Additionally, the lubricant(s) in the enteric coat can be the same or different than the copolymer(s) in a seal coat, if present, and can be one or more of the lubricants listed above. In one embodiment, the lubricant is talcum that is optionally micronized.

The copolymer(s) in the enteric coat can be the same or different than the copolymer(s) in a seal coat, if present, and can be one or more of the copolymer(s) listed above. In one embodiment, the enteric coat contains one or more of a methyl acrylate-methyl methacrylate-methacrylic acid copolymer (EUDRAGIT® FS 30 D), a methacrylic acid-methyl methacrylate copolymer and a methacrylic acid-ethyl acetate copolymer.

The enteric polymers used in the composition described herein can be modified by mixing or layering with other known coating products that are not pH sensitive. Examples of such coating products include ethyl cellulose, hydroxylpropyl cellulose, neutral methacrylic acid esters with a small portion of trimethylammonioethyl methacrylate chloride, sold currently under the trade names EUDRAGIT® RS and EUDRAGIT® RL; a neutral ester dispersion without any functional groups, sold under the trade names EUDRAGIT® NE 30 D; and other pH independent coating products.

The total amount of the copolymer(s) and/or polymer(s) in the enteric coating can range, for example, from about 25% w/w to about 100% w/w, based on the weight of the enteric coating.

If present in an enteric coating, the total amount of lubricant(s) in the enteric coating can range, for example, from a positive amount greater than 0% w/w to about 58% w/w, based on the weight of the enteric coating.

If present in an enteric coating, the total amount of filler(s) in the enteric coating can range, for example, from a positive amount greater than 0% w/w to about 5.0% w/w, based on the weight of the enteric coating.

Solvents for applying the coating materials, can be, but are not limited to, water, acetone, hexane, ethanol, methanol, propanol, isopropanol, butanol, isobutanol, sec-butanol, tert-butanol, dichlormethane, trichloromethane, chloroform, and the like.

Coatings can be applied by any known means, including spraying. In some embodiments, the compositions are coated or partially coated with one or more seal coatings, for example one, two, three or more seal coatings. In some embodiments, the compositions are coated or partially coated with one or more enteric coatings, for example one, two, three or more enteric coatings. In some embodiments, the compositions are coated with one or more seal coatings and one or more enteric coatings. In some embodiments, the compositions are coated with one seal coating and one enteric coating.

In a specific embodiment, the pharmaceutical composition is a tablet, for example, the tablet set forth in Table 1 and further coated with a seal coating solution and an enteric coating solution according to Formula A as set forth in Table 2 (See Example 1, Section 6.1 infra).

In a specific embodiment, the pharmaceutical composition is a tablet, for example, the tablet set forth in Table 1 and further coated with a seal coating solution and an enteric coating solution according to Formula B as set forth in Table 2 (See Example 1, Section 6.1 infra).

In a specific embodiment, the pharmaceutical composition is the same as in TECFIDERA®. In another specific embodiment, the pharmaceutical composition contains different fumarates from those fumarates in FUMADERM®.

In one embodiment, the pharmaceutical composition is in the form of a tablet or a capsule. In one embodiment, the pharmaceutical composition is in the form of an enterically coated tablet. In one embodiment, the pharmaceutical composition is in the form of an enterically coated microtablet.

A specific embodiment of a pharmaceutical composition is as follows. The DMF drug product is formulated as enteric-coated microtablets in gastro-resistant, hard gelatin, delayed-release capsules for oral administration. The capsules contain either 120 or 240 mg DMF. Excipients for the manufacturing of the enteric-coated microtablets include microcrystalline cellulose, croscarmellose sodium, talc, colloidal silicon dioxide, magnesium stearate, triethyl citrate, methacrylic acid copolymer Type A, methacrylic acid copolymer dispersion, simethicone (30% emulsion), sodium laurel sulfate, and polysorbate 80. Excipients for the manufacturing of the capsule shell include gelatin, titanium dioxide (E171), FD&C Blue 1; brilliant Blue FCF (E133), and yellow iron oxide (E172). The capsule print (black ink) contains black iron oxide (E172).

### 5.3 Dosing Regimens

This disclosure provides dosing regimens for administering dimethyl fumarate (DMF). The administering is done orally.

The invention provides a method for treating MS including an up-titration protocol, in which a starting dose of 120 mg DMF is used to initiate DMF therapy, and then the total daily dose of DMF is thereafter increased by 120 mg every 2 weeks, until the maintenance dose of 480 mg daily (e.g., 240 mg BID) is reached. Thus, a starting dose of 120 mg DMF is administered daily for 2 weeks, followed by 240 mg DMF daily for 2 weeks, followed by 360 mg DMF daily for 2 weeks, followed by 480 mg DMF daily as a maintenance dose.

In a specific embodiment, a starting dose of 120 mg DMF is administered daily for 2 weeks, followed by 120 mg DMF BID for 2 weeks, followed by 360 mg DMF daily for 2 weeks, followed by 240 mg DMF BID as a maintenance dose; wherein the 360 mg DMF daily is 240 mg DMF in the morning and 120 mg DMF in the evening (or about 12 h apart).

In a specific embodiment, the starting dose is 120 mg DMF once daily given in the morning for the first two weeks.

In a specific embodiment, administering of DMF as per a dosing regimen above results in reduced incidence of gastrointestinal adverse events compared to a dosing regimen in which the dose is increased to 240 mg DMF BID immediately following a starting dose of 120 mg DMF BID for one week.

The daily dosage form can be administered once daily, or divided into multiple dose administrations, for example, once, twice, thrice, four time, five times, or six times per day. The maintenance dosage of 480 mg DMF daily can be administered, for example, for at least one, two, three, four, five, six, or seven days, or for at least one, two, three, or four weeks, or for at least one, two, three, four, five, six, seven, eight, nine, ten, eleven, twelve months, or longer.

In some embodiments, a method described herein comprises orally administering a dosage form that provides a total amount of about 60 mg to about 480 mg of DMF daily. The dosage form can, for example, contain a total amount of DMF effective for treatment, prophylaxis, or amelioration of multiple sclerosis in a subject. The amount of DMF in the pharmaceutical composition can range, but is not limited to, a total amount of about 60 mg to about 480 mg DMF, about 60 mg to about 420 mg DMF, about 60 mg to about 360 mg DMF, about 60 mg to about 240 mg DMF, about 60 mg to about 220 mg DMF, about 60 mg to about 200 mg DMF, about 60 mg to about 180 mg DMF, about 60 mg to about 160 mg DMF, about 60 mg to about 140 mg DMF, about 60 mg to about 120 mg DMF, about 60 mg to about 100 mg DMF, about 60 mg to about 80 mg DMF, about 80 mg to about 480 mg DMF, about 100 mg to about 480 mg DMF, about 120 mg to about 480 mg DMF, about 140 mg to about 480 mg DMF, about 160 mg to about 480 mg DMF, about 180 mg to about 480 mg DMF, about 200 mg to about 480 mg DMF, about 220 mg to about 480 mg DMF, about 240 mg to about 480 mg DMF, about 300 mg to about 480 mg DMF, about 360 mg to about 480 mg DMF, about 400 mg to about 480 mg DMF, about 450 mg to about 500 mg DMF, about 480 mg to about 500 mg DMF, about 80 to about 400 mg DMF, about 100 to about 300 mg DMF, about 120 to about 180 mg DMF, or about 140 mg to about 160 mg DMF.

The dosage form can contain, but is not limited to, a total amount of DMF of about 60 mg DMF, about 80 mg DMF, about 100 mg DMF, about 120 mg DMF, about 140 mg DMF, about 160 mg DMF, about 180 mg DMF, about 200 mg DMF, about 220 mg DMF, about 240 mg DMF, about 260 mg DMF, about 280 mg DMF, about 300 mg DMF, about 320 mg DMF, about 340 mg DMF, about 360 mg DMF, about 380 mg DMF, about 400 mg DMF, about 420 mg DMF, about 450 mg DMF, or about 480 mg DMF.

For the treatment of multiple sclerosis (e.g., relapsing forms of multiple sclerosis such as RR-MS), the dosage form administered to the subject can be a capsule with microtablets containing DMF as the only active ingredient or microtablets consisting essentially of DMF. When the maintenance amount of 480 mg DMF is administered per day, the subjects can receive the effective amount, e.g., 240 mg DMF BID, in the form of two capsules a day, to be taken orally.

In certain embodiments, the DMF is administered with food. In other embodiments, the DMF is administered without food. For those subjects who experience GI or flushing side effects, administering DMF with food can improve tolerability. In one embodiment, administering DMF with food reduces the incidence of flushing.

In a specific embodiment, a NSAID (e.g., aspirin) is administered concurrently, before, and/or after administration of DMF. In a specific embodiment, 325 mg non-enteric coated aspirin is administered 30 minutes prior to DMF dosing, in order to reduce the occurrence and severity of flushing. Some subjects who experience flushing with gastrointestinal side effects may reduce the maintenance dose to 120 mg DMF BID temporarily. In a specific embodiment, the effective dose of 240 mg DMF BID is resumed within a month.

In one embodiment, subjects administered a dosage form described above may take one or more non-steroidal anti-inflammatory drugs (e.g., aspirin) before (for example, 10 minutes to an hour, e.g., 30 minutes before) taking the dosage form described above. In one embodiment, the subject administered the dosage form takes the one or more non-steroidal anti-inflammatory drugs (e.g., aspirin) to reduce flushing. In another embodiment, the one or more non-steroidal anti-inflammatory drugs is selected from a group consisting of aspirin, ibuprofen, naproxen, ketoprofen, celecoxib, and combinations thereof. The one or more non-steroidal anti-inflammatory drugs can be administered in an amount of about 50 mg to about 500 mg before taking the dosage form described above. In one embodiment, a subject takes 325 mg aspirin before taking each dosage form described above.

In some embodiments, subjects orally administered one or more non-steroidal anti-inflammatory drugs (e.g., aspirin) before taking the dosage form described above exhibit the same pharmacokinetic properties (e.g., Cₘₐₓ and AUC) as subjects orally administered the dosage form described above without administering one or more non-steroidal anti-inflammatory drugs (e.g., aspirin).

In one embodiment for administration of the maintenance dose of 480 mg daily, subjects with multiple sclerosis are administered a capsule containing 240 mg DMF, twice daily for a total daily dose of 480 mg, wherein the capsule contains multiple microtablets comprising about 43% w/w to about 95% w/w (e.g., from about 50% to about 80% w/w) DMF, by weight of the microtablets without any coatings. In one embodiment, the microtablets are first coated with a seal coat and then coated with an enteric coat.

### 5.4 Patient Populations

As used herein, the terms “patient” and “subject” can be used interchangeably. DMF as described herein is administered to a subject in need thereof, a subject having MS. In a specific embodiment, said subject has been diagnosed as having MS by a medical practitioner.

In some embodiments, the form of the multiple sclerosis is relapsing remitting, secondary progressive, primary progressive, or chronic progressive multiple sclerosis. In one embodiment, the patient with multiple sclerosis is a patient with a relapsing form of MS. In a specific embodiment, the patient has relapsing-remitting MS (RR-MS). In another specific embodiment, the patient has secondary-progressive MS (SP-MS). In yet another specific embodiment, the patient has progressive-relapsing MS (PR-MS).

In one embodiment, the patient is not pregnant. In another embodiment, the patient is not a nursing mother.

In one embodiment, the patient has no hypersensitivity to DMF administered in the methods described herein. In a further embodiment, the patient has no hypersensitivity to DMF or does not know about his hypersensitivity to DMF.

In one embodiment, the patient is not treated simultaneously with DMF and any immunosuppressive or antineoplastic medication. In certain embodiments, the patient is not treated simultaneously with DMF and any immunosuppressive or immunomodulatory medications or natalizumab. In certain embodiments, the patient is not treated simultaneously with DMF and any medications carrying a known risk of causing progressive multifocal leukoencephalopathy (PML).

In one embodiment, the patient has never been treated with DMF, prior to commencement of therapy in accordance with the methods disclosed herein. In another embodiment, the patient has not been treated with DMF, 1, 2, 3, 4, 6, 8, 10, or 12 months or 1, 2, 3, 5, 10, 20, 30, 40, or 50 years, prior to commencement of therapy in accordance with the methods disclosed herein.

In one embodiment, the patient has never been treated with any immunosuppressive or antineoplastic medication prior to commencement of therapy in accordance with the methods disclosed herein. In a further embodiment, the patient has not been treated with any immunosuppressive or antineoplastic medication 1, 2, 3, 4, 6, 8, 10, or 12 months or 1, 2, 3, 5, 10, 20, 30, 40, 50 years, prior to commencement of therapy in accordance with the methods disclosed herein. In another embodiment, the patient has never been treated with any immunosuppressive or immunomodulatory medications or natalizumab prior to commencement of therapy in accordance with the methods disclosed herein. In yet another embodiment, the patient has not been treated with any immunosuppressive or immunomodulatory medications or natalizumab 1, 2, 3, 4, 6, 8, 10, or 12 months or 1, 2, 3, 5, 10, 20, 30, 40, or 50 years, prior to commencement of therapy in accordance with the methods disclosed herein. In another embodiment, the patient has never been treated with any medications carrying a known risk of causing PML prior to commencement of therapy in accordance with the methods disclosed herein. In yet another embodiment, the patient has not been treated with any medications carrying a known risk of causing PML 1, 2, 3, 4, 6, 8, 10, or 12 months or 1, 2, 3, 5, 10, 20, 30, 40, or 50 years, prior to commencement of therapy in accordance with the methods disclosed herein.

In one embodiment, the immunosuppressive or antineoplastic medication is selected from one or more of: chlorambucil, melphalan, 6-mercaptopurine, thiotepa, ifodfamide, dacarbazine, procarbazine, temozolomide, hexamethylmelamine, doxorubicine, daunarubicine, idarubicin, epirubicin, irinotecan, methotrexate, etoposide, vincristine, vinblastine, vinorelbine, cytarabine, busulfan, amonifide, 5-fluorouracil, topotecan, mustargen, bleomycin, lomustine, semustine, mitomycin C, mutamycin, cisplatin, carboplatin, oxaliplatin, methotrexate, trimetrexate, raltitrexid, flurorodeoxyuridine, capecitabine, ftorafur, 5-ethynyluracil, 6-thioguanine, cladribine, pentostatin, teniposide, mitoxantrone, losoxantrone, actinomycin D, vindesine, docetaxel, amifostine, interferon alpha, tamoxefen, edroxyprogesterone, megestrol, raloxifene, letrozole, anastrzole, flutamide, bicalutamide, retinoic acids, arsenic trioxide, rituximab, CAMP ATH-1, mylotarg, mycophenolic acid, tacrolimus, glucocorticoids, sulfasalazine, glatiramer, fumarate, laquinimod, FTY-720, interferon tau, daclizumab, infliximab, IL1O, anti-IL2 receptor antibody, anti-IL-12 antibody, anti-IL6 receptor antibody, CDP-571, adalimumab, entaneracept, Ieflunomide, anti-interferon gamma antibody, abatacept, fludarabine, cyclophosphamide, azathioprine, cyclosporine, intravenous immunoglobulin, 5-ASA (mesalamine), and a β-interferon.

In one embodiment, the immunosuppressive or immunomodulatory medication is selected from one or more of: calcinerurin inhibitors, corticosteroids, cytostatics, nitrosoureas, protein synthesis inhibitors, dactinomycin, anthracyclines, mithramycin, polyclonal ntibodies such as atgum and thymoglobulin, monoclonal antibodies such as muromonab-CD3, and basiliximab, ciclosporin, sirolimus, rapamycin, γ-interferon, opioids, TNF binding proteins, TNF-α binding proteins, etanercept, mycophenolate, fingolimode, and myriocin.

In one embodiment, the patient being treated in accordance with the methods described herein has no identified systemic medical condition resulting in a compromised immune system function.

In one embodiment, the patient has been free of an immunosuppressant or immunomodulatory therapy for the patient's lifetime, or since diagnosis with MS, for example a relapsing form of MS.

### 6. EXAMPLES

### 6.1 Example 1: Compositions of Dimethyl Fumarate

A pharmaceutical composition comprising dimethyl fumarate was prepared as 2 millimeter enteric coated microtablets in a size 0 hard gelatin capsule. Each capsule contained either 120 mg dimethyl fumarate or 240 mg dimethyl fumarate.

### 6.1.1 Uncoated Core Microtablet Formulations

Dimethyl fumarate (DMF), croscarmellose sodium, talc, and colloidal silica anhydrous were mixed together to form a blend according to the amounts as described in Table 1. The blend was then passed through a screen (e.g., screen with 800 micron aperture) and microcrystalline cellulose (PROSOLV SMCC® HD90) was added to the blend and mixed. Magnesium stearate was added to the blend and the blend was remixed. The resulting blend was then compressed on a suitable rotary tablet press equipped with 16 multi-tip tooling having 2 mm round concave tips.

Table 1 below provides the weight percentages of ingredients present in two types of microtablets, 120 mg DMF and 240 mg DMF, respectively, made using the method described above. Microtablets were coated as described in Section 6.1.2 and then loaded into capsules. A size 0 capsule containing microtablets made with blend A contains about 120 mg of DMF, whereas the same size capsule containing microtablets made with blend B contains about 240 mg of DMF.

TABLE 1

| | Blend A | | Blend B, % w/w | |
|---|---|---|---|---|
| | | Per | | Per |
| | Composition, | microtablet | Composition, | microtablet |
| Ingredients | % w/w | content (mg) | % w/w | content (mg) |
| DMF | 42 | 3.1 | 65 | 5.2 |
| Croscarmellose | 5 | 0.37 | 5 | 0.4 |
| sodium | | | | |
| Prosolv | — | — | 29 | 2.3 |
| SMCC ® | | | | |
| HD90 | | | | |
| Avicel PH200 | 44 | 3.2 | — | — |
| Magnesium | 1.7 | 0.12 | 0.5 | 0.04 |
| Stearate | | | | |
| Talc | 6.6 | 0.49 | — | — |
| Silica colloidal | 0.86 | 0.067 | 0.6 | 0.048 |
| anhydrous | | | | |
| Total | 100 | 7.4 | 100 | 8 |

### 6.1.2 Microtablet Coating Formulations

The microtablets were coated with two coatings; a seal coating, followed by an enteric coating, using the seal coating formulation and enteric coating formulation of Formula A and Formula B as described in Table 2. The seal coating formulation was a solvent-based formulation which used isopropyl alcohol as a solvent, and the enteric coating formulation was based on methyl acrylic acid copolymer dispersion and provided effective enteric protection. The enteric coating formulation contained methacrylic acid copolymer dispersion and talc in addition to an antifoaming agent (simethicone). The coated microtablets were then loaded into size 0 hard gelatin capsules.

TABLE 2

| | | |
|---|---|---|
| | Coating Formula A, | Coating Formula B, |
| Ingredients | % w/w | % w/w |

Seal Coating Formulation

| | | |
|---|---|---|
| Methacrylic acid | 44.0 | 51.7 |
| copolymer, | | |
| Type A² | | |
| Triethyl citrate | 1.1 | 1.3 |
| Isopropyl alcohol¹ | 54.9 | 47.1 |

Enteric Coating Formulation

| | | |
|---|---|---|
| Methacrylic acid | 36.9 | 44.3 |
| copolymer | | |
| dispersion² | | |
| Triethyl citrate | 2.2 | 2.6 |
| Talc, micronized | 4.6 | 5.5 |
| Simethicone | 0.2 | 0.2 |
| (30% emulsion) | | |
| Purified water¹ | 59.0 | 47.3 |

¹Ingredients are removed during the process
²Methacrylic acid copolymer Type A and methacrylic acid copolymer dispersion are anionic copolymers comprising methacrylic acid and methacrylate and are the primary substances in various EUDRAGIT ® formulations, which mediate pH-dependent release of compounds.

### 6.2 Example 2: Formation of Capsules Containing Microtablets

Dimethyl fumarate, croscarmellose sodium, talcum and colloidal silicon anhydrous are mixed together to form a blend according to the amounts described in Table 3 below. The blend is passed through a screen. A suitable grade of microcrystalline cellulose, for example, PROSOLV SMCC® 90 or PROSOLV SMCC® HD90 is added to the blend and mixed. Magnesium stearate is added to the blend and the blend is remixed.

The blend is then compressed on a suitable rotary tablet press equipped with multi-tip tooling (e.g., a 16 multi-tip tooling) having 2 mm round concave tips. The resulting 2 mm sized microtablets are coated with a solution of methacrylic acid-methyl methacrylate copolymer and triethyl citrate in isopropanol (see amounts in Table 3 below). The coated microtablets are then coated with a second layer of coating consisting of methacrylic acid-ethylacrylate copolymer, polysorbate 80, sodium lauryl sulfate, triethyl citrate, simethicone, and talcum micronized suspended in water (see amounts in Table 3 below).

The desired amount of coated microtablets are encapsulated in a two piece hard gelatin capsule using a capsule machine. For example, coated microtablets are encapsulated in a capsule such that the amount of dimethyl fumarate is about 240 mg per capsule.

In Table 3 below, % w/w is based on the total weight of the coated microtablet (e.g., in this table, % w/w includes the weight contributions of the coatings).

TABLE 3

| | Net capsule content, % w/w of the capsule components | | | | | | | | | |
|---|---|---|---|---|---|---|---|---|---|---|
| | Example No. | | | | | | | | | |
| Ingredients | 1 | 2 | 3 | 4 | 5 | 6 | 7 | 8 | 9 | 10 |
| Dimethyl | 43.01 | 72.30 | 58.40 | 54.08 | 83.60 | 73.90 | 39.50 | 65.00 | 33.90 | 42.00 |
| fumarate | | | | | | | | | | |
| Croscarmellose | 1.26 | 0.33 | 3.72 | 4.17 | 0.46 | 0.89 | 4.43 | 4.00 | 4.24 | 3.00 |
| sodium | | | | | | | | | | |
| Microcrystalline | 41.82 | 15.91 | 17.31 | 23.57 | 7.00 | 9.42 | 31.31 | 13.66 | 37.18 | 35.79 |
| Cellulose | | | | | | | | | | |
| Magnesium | 1.05 | 0.25 | 0.69 | 0.41 | 0.26 | 0.63 | 1.32 | 0.40 | 1.41 | 0.48 |
| Stearate | | | | | | | | | | |
| Silica colloidal | 1.21 | 0.22 | 0.78 | 0.97 | 0.43 | 0.29 | 0.69 | 0.40 | 0.73 | 0.68 |
| anhydrous | | | | | | | | | | |
| Methacrylic | 1.01 | 1.27 | 0.98 | 1.51 | 0.11 | 1.66 | 1.87 | 1.21 | 1.55 | 1.32 |
| acid methyl | | | | | | | | | | |
| acrylate | | | | | | | | | | |
| copolymer | | | | | | | | | | |
| Methacrylic | 6.23 | 4.98 | 11.12 | 8.97 | 4.34 | 8.21 | 9.93 | 7.72 | 9.04 | 9.98 |
| acid ethyl | | | | | | | | | | |
| acrylate | | | | | | | | | | |
| copolymer | | | | | | | | | | |
| Triethyl citrate | 1.61 | 1.74 | 2.33 | 2.12 | 0.97 | 1.67 | 2.31 | 2.09 | 2.15 | 2.32 |
| Talc | 2.56 | 2.81 | 4.32 | 3.90 | 2.65 | 3.06 | 8.32 | 5.30 | 9.46 | 4.12 |
| Simethicone | 0.03 | 0.02 | 0.03 | 0.05 | 0.02 | 0.03 | 0.02 | 0.02 | 0.06 | 0.02 |
| polysorbate 80 | 0.15 | 0.11 | 0.24 | 0.20 | 0.11 | 0.18 | 0.22 | 0.14 | 0.21 | 0.21 |
| Sodium Lauryl | 0.06 | 0.06 | 0.08 | 0.07 | 0.05 | 0.06 | 0.08 | 0.06 | 0.06 | 0.08 |
| sulfate | | | | | | | | | | |

### 6.3 Example 3: Formation of Microtablets

Dimethyl fumarate, croscarmellose sodium, talcum and colloidal silicon anhydrous were mixed together to form blends 1, 2, 4, 5, and 6 according to the amounts described in Table 4 below. Each blend was passed through a screen. Microcrystalline cellulose (PROSOLV SMCC® HD90) was added to the blends according to the amounts in Table 4 and mixed. Magnesium stearate was then added to each blend and the blend was remixed. Each blend was then compressed on a suitable rotary tablet press equipped with 16 multi-tip tooling having 2 mm round concave tips.

Blends 3, 7, 8, and 9 can be made using the same method as described above.

TABLE 4

| | Percent w/w Composition of the Core Microtablet | | | | | | | | |
|---|---|---|---|---|---|---|---|---|---|
| | Blend | Blend | Blend | Blend | Blend | Blend | Blend | Blend | Blend |
| Ingredient | 1 | 2 | 3 | 4 | 5 | 6 | 7 | 8 | 9 |
| Dimethyl fumarate | 42.0 | 42.0 | 50.0 | 60.0 | 65.0 | 70.0 | 75.0 | 85.0 | 95.0 |
| Croscarmellose sodium | 5.0 | 5.0 | 3.0 | 5.0 | 5.0 | 5.0 | 1.0 | 1.0 | 0.4 |
| Microcrystalline | 44.0 | 50.0 | 43.0 | 32.0 | 28.3 | 23.0 | 22.0 | 13.0 | 4.0 |
| Cellulose | | | | | | | | | |
| Magnesium Stearate | 1.7 | 1.7 | 0.5 | 1.7 | 0.5 | 1.3 | 0.4 | 0.4 | 0.4 |
| Silica colloidal | 0.9 | 1.2 | 1.5 | 1.0 | 1.2 | 0.9 | 0.6 | 0.5 | 0.5 |
| anhydrous | | | | | | | | | |
| Talc | 6.6 | — | 2.0 | — | — | — | 1.0 | — | — |
| total | 100 | 100 | 100 | 100 | 100 | 100 | 100 | 100 | 100 |

### 6.4 Example 4: Compacts Containing 42% w/w, 60% w/w, and 70% w/w Dimethyl Fumarate and Control Compacts

Dimethyl fumarate, croscarmellose sodium, and silica colloidal anhydrous were blended together to form a blend. The blend was passed through a screen. A suitable grade of microcrystalline cellulose was added to the screened blend and the blend was mixed. A suitable grade of microcrystalline cellulose, is, for example PROSOLV SMCC® 90, having an average particle size by laser diffraction of about 60 μm and a bulk density ranging from about 0.38 to about 0.50 g/cm³. Magnesium stearate was added to the mixed blend and remixing was effected.

The respective blended materials were compressed on a suitable rotary press (e.g., a rotary tablet press) to form compacts (10 mm cylindrical compacts).

Table 5 provides percentages for representative compacts made by this process.

TABLE 5

| Ingredients | 42% | 60% | 70% |
|---|---|---|---|
| Dimethyl fumarate | 42 | 60 | 70 |
| Croscarmellose sodium | 5.0 | 5.0 | 5.0 |
| Microcrystalline Cellulose | 50 | 32 | 23 |
| Magnesium Stearate | 1.7 | 1.7 | 1.7 |
| Silica colloidal anhydrous | 1.2 | 1.0 | 0.9 |

### 6.5 Example 5: Compositions Containing 65% w/w, 95% w/w, and 99.5% w/w Dimethyl Fumarate

Four DMF-containing blends were prepared according to the method as described in Example 4 above with the amounts as described in Table 6 below.

TABLE 6

| | Composition, % by weight | | | |
|---|---|---|---|---|
| Ingredients | Blend 93 | Blend 97 | Blend 104 | Blend 108 |
| Dimethyl fumarate | 65 | 95 | 99.5 | 95 |
| Prosolv SMCC 90 | 28.9 | 2 | — | 2 |
| Croscarmellose | 5 | 2 | — | 2 |
| Sodium | | | | |
| Silica colloidal, | 0.6 | 0.6 | — | 0.6 |
| anhydrous | | | | |
| Magnesium | 0.5 | 0.4 | 0.5 | 0.4 |
| stearate | | | | |
| Particle size of | 14% < | 14% < | 15% < | 84% < |
| dimethyl fumarate | 250μ | 250μ | 250μ | 250μ |
| Flodex(mm) | 4 | 4 | 4 | 6 |
| Bulk density(g/ml) | 0.66 | 0.66 | 0.74 | 0.69 |
| Tapped | 0.79 | 0.78 | 0.83 | 0.83 |
| density(g/ml) | | | | |
| Compressibility, % | 17 | 16 | 17 | 17 |

### 6.6 Example 6. A Multicenter, Treatment-Blind Phase IV Study to Evaluate Whether 6-Week Up-Titration in Tecfidera® Dose is Effective in Reducing the Incidence of Gastrointestinal Adverse Events in Multiple Sclerosis Patients

### 6.6.1 List of Abbreviations and Definitions

The following abbreviations and definitions are used in this study protocol:

| | |
|---|---|
| AE | adverse event |
| BID | twice daily |
| CBC | complete blood count |
| CRF | case report form |
| DMF | dimethyl fumarate |
| EDC | electronic data capture |
| GCP | Good Clinical Practice |
| GI | gastrointestinal |
| GLM | general linear model |
| GSRS | Gastrointestinal Symptom Rating Scale |
| ICF | informed consent form |
| ICH | International Conference on Harmonisation |
| IFN-β | interferon-beta |
| ITT | intent-to-treat |
| IXRS | Interactive Voice and Web Response System |
| MS | multiple sclerosis |
| MSIS-29 | Multiple Sclerosis Impact Scale-29 Items |
| PHI | protected health information |
| SAE | serious adverse event |
| SUSAR | suspected unexpected serious adverse reactions |
| US | United States |
| WPAI-MS | Work Productivity and Activity Impairment questionnaire- |
| | Multiple Sclerosis, Version 2.0 |

### 6.6.2 Synopsis

This is a brief summary of the study protocol.

The study is designed to generate clinical evidence that supports whether a slower up-titration (>1 week) reduces gastrointestinal (GI) adverse events (AEs) in patients initiating DMF treatment.

### 6.6.2.1. Study Objectives

Primary

The primary objective of the study is to assess whether a 6 week titration (compared to a 1 week titration) is effective in reducing the incidence of DMF-related GI AEs in subjects with MS.

Secondary

The secondary objective of this study is to assess whether a 6-week titration (compared with a 1-week titration) is effective in reducing the average severity in GI symptoms over 12 weeks of DMF treatment in this study population.

Exploratory

The exploratory objective of this study is to assess whether a 6-week titration (compared with a 1-week titration) is effective in reducing the AE-related discontinuations in this study population.

### 6.6.2.2. Study Endpoints Primary

The primary endpoint is the proportion of subjects with a worsening in severity of GI AEs, defined as a positive average change from baseline to the end of DMF treatment in the Gastrointestinal Symptom Rating Score (GSRS).

Secondary

The secondary endpoints of the study are the:
Average GSRS scores in GI severity over the 12 weeks of DMF treatment
Time to first worsening in GI severity from baseline
Time to recovery to baseline score from the last worsening in GSRS score

Exploratory

The exploratory endpoint is the proportion of subjects who discontinued the study due to GI AEs at Week 14.

### 6.6.2.3. Study Design

This is a randomized, multicenter, treatment-blinded, parallel-group, Phase 4 study to evaluate the effect of 6 week up-titration of DMF treatment on the severity of GI AEs in subjects with MS. This is a multicenter study to be conducted in the US and the European Union.

Approximately 300 subjects will be enrolled in the study with 150 subjects in each treatment arm. Eligible subjects will be randomly assigned in a 1:1 ratio to receive either the standard 1-week titration or a 6-week titration where the total daily dose of DMF is increased by 120 mg every 2 weeks. All subjects will receive single blind placebo BID during a 2-week baseline period (Weeks 1 and 2) to establish a reliable baseline for the GSRS assessment. Following the placebo baseline period, subjects will receive DMF for 12 weeks according to their randomized treatment (Weeks 3 through 14).

Following a 28-day screening period, eligible subjects will report to the clinic at the beginning of the 2-week baseline period and at Weeks 4, 8, and 14 (end-of-treatment visit). A safety follow-up telephone interview will be conducted 2 weeks (±5 days) after the final dose of study treatment. Subjects who discontinue the study will be asked to complete the end-of-treatment assessments and safety follow-up. Discontinued subjects will not be replaced.

### 6.6.2.4. Study Population

This study will be conducted in subjects with a diagnosis of MS consistent with the locally labeled indication for Tecfidera. Subjects should be between 18 and 65 years of age, inclusive; have had no prior treatment with oral DMF; and had a recent (within the previous 6 months) complete blood count that does not preclude participation in this study, in the judgement of the Investigator.

### 6.6.2.5. Treatment Groups

Subjects will be randomly assigned in a 1:1 ratio (150 subjects per group) to either the standard 1-week titration arm or the 6-week titration arm.

All subjects will receive 2 weeks of single-blind placebo BID to establish a reliable baseline for assessment of the GSRS.

Following the placebo baseline period, subjects will receive one of the following DMF treatments:

Group 1 (Standard treatment arm):
120 mg BID for 1 week
240 mg BID for 11 weeks

Group 2 (Slow up-titration arm):
120 mg once daily (morning dose) and placebo once daily (evening dose) for 2 weeks
120 mg BID for 2 weeks
240 mg in the morning and 120 mg in the evening for 2 weeks
240 mg BID for 6 weeks

All doses of study treatment will be taken orally and may be taken with or without food. Dose modifications are not allowed. At each study visit, study treatment will be dispensed for at-home dosing with dosing information captured in a dosing diary.

### 6.6.2.6. Visit Schedule

Clinic visits are planned at screening, start of the baseline period, and during the treatment period at Weeks 4, 8, and 14 (end of treatment). Approximately 2 weeks after the last dose of study treatment, subjects will be contacted for a safety follow up telephone interview.

### 6.6.2.7. Duration of Treatment and Follow-Up

The duration of a subject's participation will be up to 20 weeks, including a 4-week screening period, a 2-week placebo baseline period, a 12-week DMF treatment period, and a 2-week safety follow-up. Subjects who discontinue the study early will complete the same assessments specified for the Week 14 (end-of-treatment) visit.

### 6.6.2.8. Criteria for Evaluation

Efficacy

The following instruments will be used to assess the effectiveness of DMF:
Multiple Sclerosis Impact Scale (MSIS)-29: 29 questions regarding the impact of MS on day to-day life during the previous 2 weeks
Work Productivity and Activity Impairment Questionnaire-Multiple Sclerosis Version 2.0 (WPAI-MS): 6 questions regarding the effect of MS on the subject's ability to work and perform regular activities in the previous 7 days

Secondary endpoints will be evaluated for correlations between responses on the collected questionnaires and the severity of GI events.

Safety

The following will be collected to monitor and evaluate the tolerability and/or safety of DMF:
Concomitant therapies and procedures
Serious AEs (SAES)
Nonserious AEs
GSRS

### 6.6.2.9. Statistical Methods

In general, continuous variables will be presented with summary statistics (mean, standard deviation, median, range), and categorical variables will be presented with frequency distributions. All analyses will be conducted using 2-sided tests at the type I error rate (alpha level) of 0.05, unless otherwise stated.

The average change from baseline to Week 14 in GSRS rating will be computed. The proportion of subjects with a worsening, defined as an average increase in GSRS rating, in GI AEs will be estimated to obtain the rate of worsening in the 2 arms. The difference in the rate of worsening in GI AEs between the 2 arms will be analyzed using a Chi-square test and a logistic regression model to adjust the covariates and the confounding factors.

The average GI severity over the 12 weeks of DMF treatment will be analyzed using a general linear model (GLM). To adjust for drop-outs, weight per subject average GSRS score proportionate to the time on study will be applied. Further, change of the GI severity with respect to time will be analyzed for each treatment arm by summarizing the corresponding GSRS score by every week.

Time to first worsening in GSRS score from baseline and time to recovery to baseline from the last worsening in GSRS score will be analyzed using Kaplan-Meier method and Cox proportional hazards model.

### 6.6.2.10. Interim Analysis, Sample Size Determination, and Study Stopping Rules

No interim analysis will be performed.

It is estimated that a total number of approximately 300 subjects, 150 per treatment arm, will provide 90% power to detect a decrease of 20% in proportion of subjects with a worsening in GI AEs in the 6-week titration arm compared with the 1-week titration arm based on Chi-square test, assuming the worst-case binomial variance, and 5% attrition rate.

There are no study-specific stopping rules. Subjects must be withdrawn from the study if the subject withdraws consent or is unwilling or unable to comply with the protocol. It is recommended that study treatment be discontinued immediately for subjects who become pregnant. Subjects may also be withdrawn at the discretion of the Investigator or Sponsor.

### 6.6.3 Study Schematic and Schedule of Events

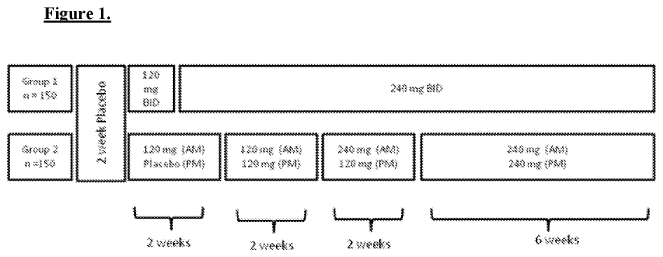

A schematic of the study design is presented in FIG. 1.

Subjects will be randomly assigned in a 1:1 ratio to either Group 1 or Group 2. Both groups will receive 2 weeks of placebo run-in to obtain baseline GI symptom scores (Weeks 1 and 2). Clinic visits will occur at Week 4 (after 2 weeks of DMF treatment), Week 8, and Week 14 (end-of-treatment visit). Approximately 2 weeks after the last dose of study treatment, a safety follow-up telephone interview will be conducted. BID=twice daily; GI=gastrointestinal.

A schedule of events is provided in Table 7.

TABLE 7
Schedule of Events

| | Screening | | | | End of | |
|---|---|---|---|---|---|---|
| | (Days −28 | Baseline | Week 4 | Week 8 | Treatment | Follow- |
| Tests and Assessments | to −1) | (Day 0) | (±4 days) | (±4 days) | (Week 14¹) | up² |
| Clinic visit | X | X | X | X | X | |
| Informed consent | X | | | | | |
| Eligibility criteria | X | | | | | |
| Demographics | X | | | | | |
| Medical History | X | X | | | | |
| MS treatment/disease history, | X | X | | | | |
| including history of relapse | | | | | | |
| Clinical laboratory tests | X | | | | | |
| (CBC, liver function tests, | | | | | | |
| and renal function tests) | | | | | | |
| Urine pregnancy test (for | X | X | X | X | X | |
| women of childbearing | | | | | | |
| potential only) | | | | | | |
| Randomization | | X | | | | |
| Blinded drug dispensed | | X | X | X | | |
| Blinded drug administration | | To be administered twice daily | | | | |
| MSIS-29 | X | | | | X | |
| WPAI-MS | X | | | | X | |
| Daily GSRS³ | | X | X | X | X | |
| Concomitant therapy | X | X | X | X | X | X |
| and procedures | | | | | | |

| | |
|---|---|
| AE and SAE recording | From time of informed consent and throughout the study, as needed |

¹Treatment period includes 2 weeks of placebo (Weeks 1 and 2) and 12 weeks of DMF treatment (Weeks 3 through 14).
²A safety follow-up telephone interview will be conducted 2 weeks (±5 days) after the final dose of study treatment.
³Training for GSRS assessments will be conducted at the site.
AE = adverse event; CBC = complete blood count; GSRS = Gastrointestinal Symptom Rating Scale; MS = multiple sclerosis; MSIS-29 = Multiple Sclerosis Impact Scale; SAE = serious adverse event; WPAI-MS = Work Productivity and Activity Impairment-Multiple Sclerosis.

### 6.6.4 Study Design

### 6.6.4.1. Study Overview

This is a randomized, multicenter, treatment-blinded, parallel-group, Phase 4 study to evaluate the effect of 6-week up-titration of DMF treatment on the severity of GI AEs in subjects with MS. The study will be conducted in the US and European Union. A schematic of the study design is presented in FIG. 1.

Approximately 300 subjects will be enrolled in the study and randomly assigned in a 1:1 ratio to receive either the standard 1-week titration or a 6-week titration where the dose of DMF is increased by 120 mg once daily every 2 weeks. All subjects will receive single-blind placebo BID during a 2-week baseline period to establish a reliable baseline for the GSRS assessment. The 12-week double-blind DMF treatment period will include the following 2 treatment arms:

Group 1 (Standard treatment arm):
120 mg BID for 1 week
240 mg BID for 11 weeks

Group 2 (Slow up-titration arm):
120 mg once daily (morning dose) and placebo once daily (evening dose) for 2 weeks
120 mg BID for 2 weeks
240 mg in the morning and 120 mg in the evening for 2 weeks
240 mg BID for 6 weeks

All doses of study treatment will be taken orally and may be taken with or without food. Dose modifications are not allowed. At each study visit, study treatment will be dispensed to each subject for at-home dosing with dosing information captured in a dosing diary.

Following a 28-day screening period, eligible subjects will report to the clinic at the beginning of the 2-week baseline period and at Weeks 4, 8, and 14 (end-of-treatment visit). A safety follow up telephone interview will be conducted 2 weeks (±5 days) after the final dose of study treatment. Subjects who discontinue the study will be asked to complete the end-of-treatment assessments and safety follow-up. Discontinued subjects will not be replaced.

### 6.6.4.2. Overall Study Duration and Follow-Up

The study period will consist of screening, a 2-week placebo baseline, a 12-week DMF treatment period, and a follow-up phone call approximately 2 weeks after the last dose of study treatment. The end of study is the last subject, last visit for final collection of data.

Screening/Baseline

At the screening visit conducted within 28 days of study entry, the subject will be asked to provide written informed consent to participate in the study. The subject's eligibility for the study will be determined based on a review of the inclusion/exclusion criteria and other assessments as indicated in Table 7.

Eligible subjects will receive single-blind placebo BID during the 2-week baseline period (Weeks 1 and 2).

Treatment

Following the 2-week placebo baseline, eligible subjects will take DMF BID for 12 weeks (Weeks 3 through 14). Subjects will return to the study site at Weeks 4, 8, and 14 (end-of-treatment visit) for assessment as indicated in Table 7.

Follow-Up

Two weeks (±5 days) after the last dose of study treatment, subjects will be interviewed by telephone as a safety follow-up for collection of information on any serious AEs (SAEs) or changes in concomitant medications or procedures.

Study Stopping Rules

There are no study-specific stopping rules.

End of Study

The end of study is last subject, last visit for final collection of data.

### 6.6.5 Study Population

### 6.6.5.1. Inclusion Criteria

To be eligible to participate in this study, candidates must meet the following eligibility criteria at the baseline visit:
1. Ability to understand the purpose and risks of the study and provide signed and dated informed consent and authorization to use protected health information (PHI) in accordance with national and local subject privacy regulations
2. Aged 18 to 65 years old, inclusive, at the time of informed consent
3. Diagnosis of MS consistent with locally labelled indication for DMF
4. No prior treatment with oral DMF
5. Female subjects of childbearing potential who are not surgically sterile must practice effective contraception during their participation in the study and be willing and able to continue contraception for 12 weeks after their last dose of DMF
6. Have had a recent (i.e., within the previous 6 months) complete blood count (CBC) that does not preclude the subject's participation in the study, in the judgment of the Investigator.

### 6.6.5.2. Exclusion Criteria

Candidates will be excluded from study entry if any of the following exclusion criteria exist at the baseline visit:
1. Are unwilling or unable to comply with study requirements, or are deemed unsuitable for study participation as determined by the Investigator
2. Have history of GI illness or treatment history that may interfere with assessment of study endpoints, as determined by the Investigator
3. Have other major comorbid conditions that preclude participation in the study, as determined by the Investigator
4. Subject is pregnant, breastfeeding, or planning a pregnancy during the study period
5. Are receiving concomitant disease-modifying therapies for MS, including but not limited to natalizumab, IFN-β, glatiramer acetate, fingolimod, alemtuzumab, teriflunomide, or laquinimod
6. History of severe allergic or anaphylactic reactions or known drug hypersensitivity
7. Current enrollment in any clinical trial or studies that may conflict with this study (e.g., health economics studies or local registries)

### 6.6.6 Enrollment and Randomization/Registration Procedures

Once the investigational site has been activated for study participation, subjects may be enrolled if they have met all the inclusion criteria in Section 6.6.5.1 and have not been excluded based on the exclusion criteria in Section 6.6.5.2.

### 6.6.6.1. Enrollment and Screening

Subjects must be consented before any screening tests or assessments are performed. At the time of consent, the subject will be considered enrolled into the study. Participating study sites are required to document all screened candidates initially considered for inclusion in this study. If a subject is excluded from the study, the reasons for exclusion will be documented in the subject's source documents and on the screening log.

### 6.6.6.2. Randomization and Registration of Subjects

Subjects will be randomly assigned to treatment group and registered at the baseline visit, after all baseline assessments have been completed and after the Investigator has verified that they are eligible per criteria in Sections 6.6.5.1 and 6.6.5.2. No subject may begin treatment prior to randomization/registration and assignment of a unique subject identification number. Any subject identification numbers that are assigned will not be reused even if the subject does not receive treatment.

Subjects will be randomly assigned to either Group 1 (standard treatment arm) or to Group 2 (slow up-titration arm) in a 1:1 ratio. Subjects who withdraw from the study may not be replaced.

### 6.6.6.3. Blinding Procedures

All study staff will be blinded to the subject treatment assignments. To maintain the study blind, it is imperative that subject treatment assignments are not shared with the subjects, their families, or any member of the study team, except the unblinded Pharmacist (or designee) and the unblinded Pharmacy Monitor.

### 6.6.7 Treatment of Subjects

### 6.6.7.1. Study Treatment Schedule and Administration

Study treatment will be dispensed to subjects at each study visit for at-home dosing. Diaries will be provided to record dosing information and will be reviewed at each visit for compliance with the treatment schedule.

All subjects will receive 2 weeks of single-blind placebo BID to establish a reliable baseline for assessment of the GSRS.

Following the 2-week placebo baseline period, DMF will be administered orally twice daily for 12 weeks as follows:

Group 1 (Standard treatment arm):
120 mg BID for 1 week
240 mg BID for 11 weeks

Group 2 (Slow up-titration arm):
120 mg once daily (morning dose) and placebo once daily (evening dose) for 2 weeks
120 mg BID for 2 weeks
240 mg in the morning and 120 mg in the evening for 2 weeks
240 mg BID for 6 weeks

Study treatment should not be crushed or chewed and the capsule contents should not be sprinkled on food. Study treatment may be taken with or without food. Administration with food may reduce the incidence of flushing. Dose modifications are not allowed.

Missed doses should be taken if there are at least 4 hours between the morning and evening doses. Otherwise, treatment should be continued with the next dose as planned. Doses should not be doubled to make up for missed doses.

A schematic of the study design is presented in FIG. 1.

### 6.6.7.2. Comparator or Reference Product Agents

Matching placebo will be administered BID during the 2-week baseline period and as the evening dose during Weeks 3 and 4 for subjects assigned to Group 2 (slow up-titration arm).

### 6.6.7.3. Concomitant Therapy

A concomitant therapy is any drug or substance administered between the time the subject is enrolled in the study and the safety follow-up telephone interview.

Subjects who have previously received oral DMF are not eligible for study participation. Concomitant use of disease-modifying therapies is not allowed. Such therapies include but are not limited to natalizumab, IFN-β, glatiramer acetate, fingolimod, alemtuzumab, teriflunomide, and laquinimod.

Apart from the excluded medications listed above, the use of concomitant medications is at the discretion of the Investigator. The prevailing product label should be used as a guideline.

Concomitant aspirin, which may reduce the occurrence of flushing, is permitted. However, subjects are to be advised that aspirin should not be used long-term for the management of flushing.

A concomitant procedure is any therapeutic intervention (e.g., surgery/biopsy and physical therapy) or diagnostic assessment (e.g., blood gas measurement and bacterial cultures) performed between the time the subject is enrolled in the study and the safety follow-up telephone interview.

### 6.6.8 Study Treatment Management

### 6.6.8.1. Dimethyl Fumarate

The DMF drug product is formulated as enteric-coated microtablets in gastro-resistant, hard gelatin, delayed-release capsules for oral administration. Capsules contain either 120 or 240 mg DMF. Excipients for the manufacturing of the enteric-coated microtablets include microcrystalline cellulose, croscarmellose sodium, talc, colloidal silicon dioxide, magnesium stearate, triethyl citrate, methacrylic acid copolymer Type A, methacrylic acid copolymer dispersion, simethicone (30% emulsion), sodium laurel sulfate, and polysorbate 80. Excipients for the manufacturing of the capsule shell include gelatin, titanium dioxide (E171), FD&C Blue 1; brilliant Blue FCF (E133), and yellow iron oxide (E172). The capsule print (black ink) contains black iron oxide (E172).

At the investigational sites, DMF is to be protected from light and stored at ≤30° C. (≤86° F.) in a secure location, preferably a monitored, locked cabinet with limited access.

### 6.6.8.2. Placebo and/or Comparator or Reference Product

Matching placebo will be packaged, stored, and destroyed identically to the DMF study treatment.

### 6.6.9 Withdrawal of Subjects from Study Treatment and/or the Study

A subject must permanently discontinue study treatment for any of the following reasons:
The subject becomes pregnant.
The subject withdraws consent.
The subject is unwilling or unable to comply with the protocol.
The subject experiences a medical emergency that necessitates permanent discontinuation of study treatment.
The subject experiences a medical emergency that necessitates unblinding of the subject's treatment assignment.
At the discretion of the Investigator for medical reasons.
At the discretion of the Investigator or Sponsor for noncompliance.

The reason for discontinuation of study treatment must be recorded in the subject's CRF.

Subjects who discontinue treatment must be permanently withdrawn from the study. If possible, the end-of-treatment assessments and safety follow-up telephone interview are to be performed on subjects who are withdrawn. Withdrawn subjects will not be replaced.

### 6.6.10 Efficacy Assessments

### 6.6.10.1. Clinical Efficacy Assessments

The following instruments will be used to assess the efficacy of DMF:
Multiple Sclerosis Impact Scale (MSIS)-29 Items: 29 questions regarding the impact of MS on day-to-day life during the previous 2 weeks [Gray 2009; Hobart 2001]
Work Productivity and Activity Impairment Questionnaire-Multiple Sclerosis Version 2.0 (WPAI-MS): 6 questions regarding the effect of MS on the subject's ability to work and perform regular activities in the previous 7 days [Glanz 2012]

Secondary endpoints will be evaluated for correlations between responses on the collected questionnaires and the severity of GI events.

Refer to Table 7 for the timing of assessments.

### 6.6.11 Safety Assessments

### 6.6.11.1. Clinical Safety Assessments

The following clinical assessments will be performed to assess the safety profile of DMF:
Concomitant therapies and procedures recording
AE and SAE recording
GSRS

Refer to Table 7 for the timing of assessments.

### 6.6.11.2. Laboratory Safety Assessments

Laboratory tests conducted at screening only include a CBC and liver and renal function tests according to the local product label; urine pregnancy tests will be conducted at each visit. Otherwise, no additional laboratory tests will be performed to assess the safety profile of DMF in this study.

### 6.6.12 Safety Definitions, Monitoring, and Reporting

Throughout the course of the study, every effort must be made to remain alert to possible AEs. If an AE occurs, the first concern should be for the safety of the subject. If necessary, appropriate medical intervention should be provided.

At the signing of the informed consent form (ICF), each subject must be given the names and telephone numbers of study site staff for reporting AEs and medical emergencies.

### 6.6.12.1. Definitions

Serious Pretreatment Event

A serious pretreatment event is any event that meets the criteria for SAE reporting (as defined under Serious Adverse Event) and occurs after the subject signs the ICF, but before administration of study treatment.

Adverse Event

An AE is any untoward medical occurrence in a patient or clinical investigation subject administered a pharmaceutical product and that does not necessarily have a causal relationship with this treatment. An AE can therefore be any unfavorable and unintended sign (including an abnormal laboratory finding), symptom, or disease temporally associated with the use of a medicinal (investigational) product, whether or not related to the medicinal (investigational) product.

Serious Adverse Event

An SAE is any untoward medical occurrence that at any dose:
Results in death
In the view of the Investigator, places the subject at immediate risk of death (a life threatening event); however, this does not include an event that, had it occurred in a more severe form, might have caused death
Requires inpatient hospitalization or prolongation of existing hospitalization
Results in persistent or significant disability/incapacity, or
Results in a congenital anomaly/birth defect

An SAE may also be any other medically important event that, in the opinion of the Investigator, may jeopardize the subject or may require intervention to prevent one of the other outcomes listed in the definition above. (Examples of such medical events include allergic bronchospasm requiring intensive treatment in an emergency room or convulsions occurring at home that do not require an inpatient hospitalization.)

### 6.6.12.2. Monitoring and Recording Events

Serious Pretreatment Events

A serious pretreatment event experienced by the subject after signing and dating the ICF, but before administration of study treatment is to be recorded on the SAE Form and faxed to Quintiles Lifecycle Safety within 24 hours of the study site staff becoming aware of the event (see Section 15.2.5).

Adverse Events

Any AE experienced by the subject between the time of signing the ICF and the safety follow-up telephone interview is to be recorded on the CRF, regardless of the severity of the event or its relationship to study treatment.

Serious Adverse Events

Any SAE experienced by the subject between the time of the first dose of study treatment and the safety follow-up telephone interview is to be recorded on an SAE Form, regardless of the severity of the event or its relationship to study treatment. Serious AEs must be reported to the Sponsor (or designee).

Any SAE ongoing when the subject completes the study or discontinues from the study will be followed by the Investigator until the event has resolved, stabilized, or returned to baseline status.

All Events

All events must be assessed to determine the following:
If the event meets the criteria for an SAE as defined under Serious Adverse Event.
The relationship of the event to study treatment as defined under Relationship of Events to Study Treatment.
The severity of the event as defined under Severity of Events.

Immediate Reporting of Serious Adverse Events

In order to adhere to all applicable laws and regulations for reporting an SAE, the study site must formally report an SAE within 24 hours of the study site staff becoming aware of the SAE. It is the Investigator's responsibility to ensure that the SAE reporting information and procedures are used and followed appropriately.

Reporting Information for SAEs
Any SAE that occurs between the time that the subject has signed informed consent and the
safety follow-up telephone interview must be reported within 24 hours of the study site staff
becoming aware of the event.
A report must be submitted regardless of the following:
Whether or not the subject has undergone study-related procedures
Whether or not subject has received study treatment
The severity of the event
The relationship of the event to study treatment

Deaths

Death is an outcome of an event. The event that resulted in death should be recorded and reported on the appropriate CRF. All causes of death must be reported as SAEs. The Investigator should make every effort to obtain and send death certificates and autopsy reports to Quintiles Lifecycle Safety.

### 6.6.12.3. Safety Classifications

Relationship of Events to Study Treatment

The following definitions should be considered when evaluating the relationship of AEs and SAEs to the study treatment:

Relationship of Event to commercial drug

| | |
|---|---|
| Not related | An AE will be considered “not related” to the use of the investigational drug if there is |
| | not a possibility that the event has been caused by the product under investigation. |
| | Factors pointing toward this assessment include, but are not limited to: the lack of |
| | reasonable temporal relationship between administration of the drug and the event, the |
| | presence of a biologically implausible relationship between the product and the adverse |
| | event (e.g., the event occurred before administration of drug), or the presence of a more |
| | likely alternative explanation for the adverse event. |
| Related | An AE will be considered “related” to the use of the investigational drug if there is a |
| | possibility that the event may have been caused by the product under investigation. |
| | Factors that point toward this assessment include, but are not limited to: a positive |
| | rechallenge, a reasonable temporal sequence between administration of the drug and the |
| | event, a known response pattern of the suspected drug, improvement following |
| | discontinuation or dose reduction, a biologically plausible relationship between the drug |
| | and the AE, or a lack of an alternative explanation for the AE. |

Severity of Events

The following definitions should be considered when evaluating the severity of AEs and SAEs:

Severity of Event

| | |
|---|---|
| Mild | Symptom(s) barely noticeable to subject or does not make subject uncomfortable; does |
| | not influence performance or functioning; prescription drug not ordinarily needed for |
| | relief of symptom(s) but may be given because of personality of subject. |
| Moderate | Symptom(s) of a sufficient severity to make subject uncomfortable; performance of daily |
| | activity is influenced; subject is able to continue in study; treatment for symptom(s) may |
| | be needed. |
| Severe | Symptom(s) cause severe discomfort; symptoms cause incapacitation or significant |
| | impact on subject's daily life; severity may cause cessation of treatment with study |
| | treatment; treatment for symptom(s) may be given and/or subject hospitalized. |

Expectedness of Events

Expectedness of all AEs will be determined according to the prevailing product label.

### 6.6.12.4. Prescheduled or Elective Procedures or Routinely Scheduled Treatments

A prescheduled or elective procedure or a routinely scheduled treatment will not be considered an SAE, even if the subject is hospitalized; the study site must document all of the following:
The prescheduled or elective procedure or routinely scheduled treatment was scheduled (or was on a waiting list to be scheduled) prior to obtaining the subject's consent to participate in the study.
The condition requiring the prescheduled or elective procedure or routinely scheduled treatment was present before and did not worsen or progress in the opinion of the Investigator between the subject's consent to participate in the study and the time of the procedure or treatment.
The prescheduled or elective procedure or routinely scheduled treatment is the sole reason for the intervention or hospital admission.

### 6.6.13 Statistical Methods and Determination of Sample Size

### 6.6.13.1. Primary Objective and Endpoint

The primary objective of the study is to assess whether a 6-week titration (compared to a 1 week titration) is effective in reducing the incidence of DMF-related GI AEs in subjects with MS.

The primary endpoint is the proportion of subjects with a worsening in severity of GI AEs, defined as a positive average change from baseline to the end of DMF treatment (Week 14) in the GSRS score.

### 6.6.13.2. Secondary Objectives and Endpoints

The secondary objective of this study is to assess whether a 6-week titration (compared with a 1 week titration) is effective in reducing the average severity in GI symptoms over 12 weeks of DMF treatment in this study population.

Secondary endpoints include the following:
Average GSRS scores in GI severity over the 12 weeks of DMF treatment
Time to first worsening in GI severity from baseline
Time to recovery to baseline score from the last worsening in GSRS score.

### 6.6.13.3. Exploratory Objectives and Endpoints

The exploratory objective of this study is to assess whether a 6-week titration (compared with a 1 week titration) is effective in reducing the AE-related discontinuations in this study population.

The exploratory endpoint is the proportion of subjects who discontinued the study due to GI AEs at Week 14 (following 12 weeks of DMF treatment).

### 6.6.13.4. Demography and Baseline Disease Characteristics

Demographics and background disease data will be summarized by presenting frequency distributions or summary statistics.

### 6.6.13.5. Efficacy Data

Analysis Population

The intent-to-treat (ITT) population defined below will be used for the efficacy analysis.

The ITT population is defined as all randomized subjects who receive at least 1 dose of DMF treatment (including the standard 1-week titration and 6-week titration).

General Methods of Analysis

In general, continuous variables will be presented with summary statistics (number of subjects with data, mean, standard deviation, median, and range), and categorical variables will be presented with frequency distributions which generally includes: number of subjects with data, number of those with data in each category, and the percent of those with data in each category. All analyses will be conducted using 2-sided tests at the type I error rate (a level) of 0.05, unless otherwise stated. All efficacy analyses will be conducted using the efficacy population.

Primary Endpoint Analysis

Change from baseline to Week 14 in the GSRS score will be computed. The proportion of subjects with worsening, defined as an increase in GSRS score, in GI AEs will be used to estimate the rate of worsening in the 2 arms. The difference in the rate of worsening in GI AEs will be analyzed using a chi-square test and the logistic regression model adjusting for covariates and confounding factors.

Secondary Endpoints Analysis

The average severity over the 12 weeks of DMF treatment will be analyzed using a general linear model (GLM) to adjust for covariates and confounding factors such as early discontinuation. To adjust for the early discontinuation, weight per subject average proportionate to the time on study will be applied. Time to first worsening in score from baseline and time to recovery to baseline from the last worsening in GSRS score in GI severity will be analyzed using the Kaplan-Meier method and a Cox proportional hazards model. The average change in GSRS score from the Day 0 score at various time points will be analyzed using a GLM model adjusting for covariates and confounding factors.

Exploratory Endpoints Analysis

The proportion of subjects who discontinue the study due to GI-related events from baseline to Week 14 will be summarized by treatment arm.

### 6.6.13.6. Safety Data

Analysis Population

The safety population will be defined as all subjects who received at least 1 dose of DMF treatment (including the standard 1-week titration and the 6-week titration).

Methods of Analysis

All safety data will be coded using the Medical Dictionary for Regulatory Activities and evaluated based on treatment emergence. The incidence of all AEs and SAEs will be presented by system organ class and preferred term. The incidence of all AEs will be summarized by severity and by relationship to study treatment. AEs leading to withdrawal from the study will also be summarized.

### 6.6.13.7. Interim Analyses

No interim analysis is planned.

### 6.6.13.8. Sample Size Considerations

Subjects will be randomized into a standard 1-week titration arm and a 6-week titration arm in a 1:1 allocation. A total sample size of 300, 150 per treatment arm, will provide 90% power to detect a 20% reduction in proportion of subjects with any worsening in GI severity in the 6-week titration arm compared with the standard 1-week titration arm. This calculation assumes that the proportions of subjects with any worsening in GI severity are 60% for the 1-week titration arm and 40% for the 6-week titration arm, which provides the worst-case binomial variance and therefore the largest sample size required. It also assumes an early attrition rate of 5% over the 12 weeks of DMF treatment and 5% type I error rate.

### 6.6.14 References for Example 6

Cree B A. 2014 multiple sclerosis therapeutic update. Neurohospitalist. 2014; 4(2):63-5.
Fox E, Green B, Markowitz C, et al. The effect of scheduled antibody testing on treatment patterns in interferon-treated patients with multiple sclerosis. BMC Neurol. 2014; 14: 73.
Fox R J, Miller D H, Phillips J T, et al. Placebo-controlled phase 3 study of oral BG-12 or glatiramer in multiple sclerosis. N Engl J Med. 2012; 367(12):1087-97. Erratum in: N Engl J Med. 2012 Oct. 25; 367(17):1673.
Glanz B I, Dégano I R, Rintell D J, et al. Work productivity in relapsing multiple sclerosis: associations with disability, depression, fatigue, anxiety, cognition, and health-related quality of life. Value Health. 2012; 15(8):1029-35.
Gold R, Kappos L, Arnold D L, et al. Placebo-controlled phase 3 study of oral BG-12 for relapsing multiple sclerosis. N Engl J Med. 2012a; 367(12):1098-107.
Gray O, McDonnell G, Hawkins S. Tried and tested: the psychometric properties of the multiple sclerosis impact scale (MSIS-29) in a population-based study. Mult Scler. 2009; 15(1):75-80.
Hanson K A, Agashivala N, Wyrwich K W, et al. Treatment selection and experience in multiple sclerosis: survey of neurologists. Patient Prefer Adherence. 2014; 8: 415-22.
Hobart J, Lamping D, Fitzpatrick R, et al. The Multiple Sclerosis Impact Scale (MSIS-29): a new patient-based outcome measure. Brain. 2001; 124(Pt 5):962-73.
Ruggieri S, Tortorella C, Gasperini C. Pharmacology and clinical efficacy of dimethyl fumarate (BG-12) for treatment of relapsing-remitting multiple sclerosis. Ther Clin Risk Manag. 2014; 10: 229-239.

### 7. INCORPORATION BY REFERENCE

Various references such as patents, patent applications, and publications are cited herein, the disclosures of which are hereby incorporated by reference herein in their entireties.

What is claimed is:

1. A method of treating a human patient with multiple sclerosis comprising orally administering to the patient a pharmaceutical composition comprising dimethyl fumarate (hereinafter “DMF”); wherein the administering step comprises administering a starting dose of 120 mg DMF daily for 2 weeks, followed by 240 mg DMF daily for 2 weeks, followed by 360 mg DMF daily for 2 weeks, followed by 480 mg DMF daily as a maintenance dose.

2. The method of claim 1, wherein the 240 mg DMF daily is 120 mg DMF BID.

3. The method of claim 1 or 2, wherein the 360 mg DMF daily is 240 mg DMF in the morning and 120 mg DMF in the evening.

4. The method of any of claims 1-3, wherein the 480 mg DMF daily is 240 mg DMF BID.

5. A method of treating a human patient with multiple sclerosis comprising orally administering to the patient a pharmaceutical composition comprising DMF; wherein the administering step comprises administering a starting dose of 120 mg DMF daily for 2 weeks, followed by 120 mg DMF BID for 2 weeks, followed by 360 mg DMF daily for 2 weeks, followed by 240 mg DMF BID as a maintenance dose; wherein the 360 mg DMF daily is 240 mg DMF in the morning and 120 mg DMF in the evening.

6. The method of any of claims 1-5, wherein the administering results in a reduced incidence of gastrointestinal adverse events compared to a dosing regimen in which the dose is increased to 240 mg DMF BID immediately following a starting dose of 120 mg DMF BID for one week.

7. The method of any of claims 1-6, wherein the pharmaceutical composition comprises DMF and a pharmaceutically acceptable carrier.

8. The method of claim 7, wherein the pharmaceutical composition is in the form of a tablet or a capsule.

9. The method of claim 7, wherein the pharmaceutical composition is in the form of an enterically coated tablet.

10. The method of claim 7, wherein the pharmaceutical composition is in the form of a capsule containing enterically coated microtablets.

11. The method of any of claims 1-10, wherein the pharmaceutical composition consists essentially of DMF.

12. The method of any of claims 1-10, wherein the pharmaceutical composition does not contain a fumarate salt.

13. The method of any of claims 1-10, wherein the pharmaceutical composition does not contain ethyl hydrogen fumarate salt.

14. The method of any of claims 1-10, wherein the pharmaceutical composition does not contain ethyl hydrogen fumarate calcium salt, ethyl hydrogen fumarate magnesium salt, ethyl hydrogen fumarate zinc salt, and ethyl hydrogen fumarate copper salt.

15. The method of any of claims 1-14, wherein the multiple sclerosis is a relapsing form of multiple sclerosis.
